(19) United States
(12) Patent Application Publication
(10) Pub. No.: US 2021/0346419 A1
(43) Pub. Date: Nov. 11, 2021

(54) COMPOSITIONS AND METHODS FOR DIAGNOSIS AND TREATMENT OF CONDITIONS RELATED TO THE QUALITY OF AGING AND LONGEVITY

(71) Applicant: EPITRACKER, INC., San Diego, CA (US)

(72) Inventor: Stephanie Venn-Watson, San Diego, CA (US)

(21) Appl. No.: 17/380,622

(22) Filed: Jul. 20, 2021

Related U.S. Application Data
(63) Continuation of application No. PCT/US2020/013913, filed on Jan. 16, 2020.
(60) Provisional application No. 62/795,780, filed on Jan. 23, 2019.

(51) Int. Cl. A61K 31/7076; A61P 7/06; A61P 29/00; A61P 3/10; A61P 3/06; A61P 1/16; A61P 3/00; A61K 31/155; A61K 31/192; A61K 31/708; A61K 31/7028; A61P 11/06; A61P 37/02; A61P 17/02; A61P 37/08; A23L 33/175; A23L 33/00

(52) U.S. Cl. CPC A61K 31/7076; A23V 2002/00; A61P 29/00; A61P 3/10; A61P 3/06; A61P 1/16; A61P 3/00; A61K 31/155; A61K 31/192; A61K 31/708; A61K 31/7028; A61P 11/06; A61P 37/02; A61P 17/02; A61P 37/08; A23L 33/175; A23L 33/40; A61P 7/06

(57) ABSTRACT

Compositions including metabolites that are small molecules, and salts and derivatives thereof, and methods for treatment or prophylaxis of conditions related to the quality of aging are provided, including compositions and methods for treating conditions that negatively impact longevity and the quality of aging, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

### INCORPORATION BY REFERENCE TO RELATED APPLICATIONS

This application is a continuation of PCT Application No. PCT/US2020/013913, filed Jan. 16, 2020, which published in English and which claims the benefit of U.S. Provisional Application No. 62/795,780, filed Jan. 23, 2019. Each of the aforementioned applications is incorporated by reference herein in its entirety, and is hereby expressly made a part of this specification.

### STATEMENT REGARDING FEDERALLY SPONSORED R&D

The United States Government has certain rights in this invention, under CRADA Number NCRADA-SSCPACIFIC-17-261.

### FIELD OF THE INVENTION

Compositions including metabolites that are small molecules, and salts and derivatives thereof, and methods for treatment or prophylaxis of conditions related to the quality of aging are provided, including compositions and methods for treating conditions that negatively impact longevity and the quality of aging, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

### BACKGROUND OF THE INVENTION

Aging increases the risk of health conditions that can decrease quality of life and longevity. As people age they have a higher risk of developing a suite of conditions, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems. These conditions are contributing factors to a reduced quality of life, and as such, treatments of these conditions have been proposed as a means to improve longevity and quality of life.

### SUMMARY OF THE INVENTION

Compositions and methods for treatment or prophylaxis of conditions with aging that impact quality of life or longevity are provided. These compositions comprise one or more small molecule biochemicals, derivatives of these biochemicals, or salts thereof, which may be administered in combination with other medicaments or as part of various treatment regimens as described herein. The provided compositions are effective for modulating markers of aging-associated conditions that impact quality of life or longevity. Methods are provided for administering the compositions.

Accordingly, in a generally applicable first aspect (i.e., independently combinable with any of the aspects or embodiments identified herein), a pharmaceutical composition is provided comprising: one or more small molecule metabolites, or pharmaceutically acceptable salts thereof, wherein the one or more small molecule metabolites are selected from the group consisting of one or more small molecule metabolites; and a pharmaceutically acceptable carrier.

In an embodiment of the first aspect (i.e., independently combinable with any of the aspects or embodiments identified herein), the one or more small molecules is a metabolite described herein.

In an embodiment of the first aspect (i.e., independently combinable with any of the aspects or embodiments identified herein), the composition is in a unit dosage form.

In an embodiment of the first aspect (i.e., independently combinable with any of the aspects or embodiments identified herein), the pharmaceutical composition is configured for administration of from 2.5 mg to 50 mg, per 1 kg of body weight, of the one or more small molecule metabolites or pharmaceutically acceptable salts thereof to a patient.

In an embodiment of the first aspect (i.e., independently combinable with any of the aspects or embodiments identified herein), the pharmaceutical composition is configured for administration once per day.

In an embodiment of the first aspect (i.e., independently combinable with any of the aspects or embodiments identified herein), the pharmaceutical composition comprises from 0.01 mg to 10000 mg of the one or more small molecule metabolites or pharmaceutically acceptable salts thereof.

In a generally applicable second aspect (i.e., independently combinable with any of the aspects or embodiments identified herein), use is provided of a pharmaceutical composition of the first aspect or any embodiment thereof, in the manufacture of a medicament for treatment or prophylaxis of conditions related to quality of life or longevity, wherein the conditions related to quality of life or longevity are selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

In an embodiment of the second aspect (i.e., independently combinable with any of the aspects or embodiments identified herein), the use is in the manufacture of a medicament for treatment or prophylaxis of conditions related to quality of life or longevity.

In an embodiment of the second aspect (i.e., independently combinable with any of the aspects or embodiments identified herein), the pharmaceutical composition is configured to modulate a marker of aging-associated conditions related to quality of life or longevity or a symptom of conditions related to quality of life or longevity.

In an embodiment of the second aspect (i.e., independently combinable with any of the aspects or embodiments identified herein), the marker of conditions related to quality of life or longevity is selected from the group consisting of serum or plasma small molecule metabolite concentration, red blood cell indices (i.e. hemoglobin, red blood cells), serum or plasma cholesterol, triglycerides, insulin, glucose, gamma-glutamyl transpeptidase, ferritin, or iron.

In an embodiment of the second aspect (i.e., independently combinable with any of the aspects or embodiments identified herein), the pharmaceutical composition is configured to increase a serum, red blood cell, or tissue concentration of the one or more small molecule metabolites to between 0.2 μM and 20 μM.

In a generally applicable third aspect (i.e., independently combinable with any of the aspects or embodiments identified herein), a method of treatment or prophylaxis of conditions associated with quality of life or longevity, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, and other related conditions, comprising: administering to a patient in need thereof, an effective amount of one or more small molecule metabolites, or pharmaceutically acceptable salts thereof, wherein the one or more small molecule metabolites are selected from the group described herein.

In an embodiment of the third aspect (i.e., independently combinable with any of the aspects or embodiments identified herein), the one or more small molecule metabolites or pharmaceutically acceptable salts thereof is provided as a pharmaceutical composition in a unit dosage form comprising the one or more small molecule metabolites or pharmaceutically acceptable salts thereof and a pharmaceutically acceptable carrier.

In an embodiment of the third aspect (i.e., independently combinable with any of the aspects or embodiments identified herein), the unit dosage form comprises from 0.01 mg to 10000 mg of the one or more small molecule metabolites or pharmaceutically acceptable salts thereof.

In an embodiment of the third aspect (i.e., independently combinable with any of the aspects or embodiments identified herein), the one or more small molecule metabolites described herein.

In an embodiment of the third aspect (i.e., independently combinable with any of the aspects or embodiments identified herein), the pharmaceutical composition comprises a plurality of different small molecule metabolites described herein.

In an embodiment of the third aspect (i.e., independently combinable with any of the aspects or embodiments identified herein), from 2.5 mg to 50 mg of the one or more small molecule metabolites or pharmaceutically acceptable salts thereof is administered to the patient, per 1 kg of body weight, per day.

In an embodiment of the third aspect (i.e., independently combinable with any of the aspects or embodiments identified herein), the one or more small molecule metabolites or pharmaceutically acceptable salts thereof is administered to the patient once per day.

In an embodiment of the third aspect (i.e., independently combinable with any of the aspects or embodiments identified herein), a serum, tissue, or a red blood cell membrane concentration of the one or more small molecule metabolites is increased 1.25 to 6 times above the patient's baseline levels to achieve concentrations between 0.5 μM and 20 μM.

In a generally applicable fourth aspect (i.e., independently combinable with any of the aspects or embodiments identified herein), a composition substantially as described herein is provided.

In a generally applicable fifth aspect (i.e., independently combinable with any of the aspects or embodiments identified herein), a composition substantially as described herein is provided.

In a generally applicable sixth aspect (i.e., independently combinable with any of the aspects or embodiments identified herein), a use substantially as described herein is provided.

### DESCRIPTION OF THE DRAWINGS

FIG. 1. provides data on improving hematocrit among individual dolphins while on the modified fish diet.

FIG. 2 provides a summary of 12 human cell systems, mimicking various disease states, used to evaluate selected compounds' efficacy in treating conditions related to the quality of aging and longevity (from Eurofins/DiscoverX BioMAP® Diversity ES Panel).

### DETAILED DESCRIPTION

Compositions including one or more small molecule metabolites, and associated methods for treatment of conditions related to aging, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, and other related conditions, are provided.

Aging is associated with chronic, low-grade inflammation characterized by increased circulating levels of proinflammatory cytokines, neutrophils, and progressively activated macrophages. This pro-inflammatory state is a significant risk factor for both morbidity and mortality in the elderly people (Franceschi C, Campisi J (2014) Chronic inflammation (inflammaging) and its potential contribution to age-associated diseases. J Gerontol Ser A 69:S4-S9). People who live at least 100 years are less likely to have developed a proinflammatory state with age, further supporting that chronic, low-grade inflammation impairs quality and duration of life (Vasto S, Candore G, Balistreri M, Colonna-Romano G, Grimaldi M P, Listi F et al. (2007) Inflammatory networks in ageing, age-related diseases, and longevity. Mech Ageing and Dev 128:83-91). As such, interventions to reduce inflammation of aging have been proposed to concurrently treat multiple aging-associated diseases, resulting in improved quality of life and expanded longevity.

As people age, the prevalence of anemia can increase dramatically. Comparisons of populations aged 40, 60, 80, 90 and 100 (total n=1,980) had the following prevalence of anemia: 16.1%, 19.1%, 41.1%, 46.2%, and 57.1% (Zhai Y, Yin Z X, Xu J W, Liu Y Z, Shi X M (2010) Anemia status and its relevant factors among elderly people aged above 80 years old in longevity areas in China. Chinese J of Prev Med 44:115-118). The presence of anemia and low hemoglobin concentration increased the 3-year mortality risk by 25% and decreased longevity among older people (Lyu Y, Yin Z, Luo J, Shi X, Zeng Y (2015) Zhonghua Liuxingbingxue Zazhi 36:682-686). Means to prevent and treat anemia of aging are expected to improve quality of life and expand longevity.

Elevated cholesterol with aging can negatively impact quality of life or longevity. (Kriesberg R A and Kasim S (1987) Cholesterol metabolism and aging, Am J Med 82:54-60). Elevated cholesterol, especially elevated low-density lipoprotein (LDL) cholesterol, have been identified as underlying causes of or contributors to cardiovascular disease, including atherosclerosis, which also increase in prevalence with age. Lower cholesterol levels, especially for people under 50 years old, have been associated with improved longevity (Anderson K M, Castelli W P, Levy D (1987) Cholesterol and mortality: 30 years of follow-up from the Framingham Study JAMA 257:2176-2180). Similarly, triglyceride levels can be major predicting factors for human longevity, with lower triglyceride levels present in long-lived families compared to controls (Vaarhorst A A M, Beekman M, Suchiman E H D, van Heemst D V, Houwing-Duistermaat J J, Westerndorp R G et al (2010) Lipid metabolism in long-lived families: the Leiden Longevity Study. AGE 33:219-227). Preventing and treating dyslipidemia have been highlighted as important to improve quality of life and expand longevity.

Prediabetic and diabetic conditions, including hyperglycemia and insulin resistance, result in impaired quality of life and longevity. From 1999 to 2011, the average number of years lost from diabetes has increased by 46% in men and 44% in women (Gregg E W, Zhuo X, Cheng Y J, Albright A L, Narayan K M V, Thompson T J (2014) Trends in lifetime risk and years of life lost due to diabetes in the USA, 1985-2011: A modelling study. Lancet Diab Endocrinol 2:867-874). Insulin resistance increases with advanced age, and people who live over one hundred years have lower insulin resistance compared to those who were younger (Paolisso G, Barbieri M, Rizzo M R, Carella C, Rotondi M, Bonafe M et al (2001) Low insulin resistance and preserved β-cell function contribute to human longevity but are not associated with TH-INS genes. Exp Gerontol 37:147-156). Treatments long used to treat type 2 diabetes, including metformin, have been demonstrated to expand longevity (Novelle M G, Ali A, Dieguez C, Bernier M, de Cabo R (2016) Metformin: A hopeful promise in aging research. CSH Perspectives 6:a025932), and there is an active effort to discover other compounds that may help treat hyperglycemia and insulin resistance and expand longevity.

The prevalence of chronic liver disease and accompanying cirrhosis and hepatocellular carcinoma have been increasing at an alarming rate, especially in developed countries. This increase is due primarily to nonalcoholic fatty liver disease (NAFLD) associated with the global rise in obesity and metabolic syndrome (including elevated glucose, dyslipidemia, and insulin resistance). Chronic liver disease contributes to morbidity and mortality, and by aiming to decrease liver failure, transplants, and cancer, therapeutics for liver disease can improve quality of life and expand longevity (Lim Y S, Kim W R (2008) The global impact of hepatic fibrosis and end-stage liver disease. Clinics in Liver Dis 12:733-746).

Iron overload and hyperferritinemia with aging can negatively impact quality of life or longevity. Iron accumulates with age in tissues, including the brain (Hirose W, Ikematsu K, and Tsuda R (2003) Age-associated increases in heme oxygenase-1 and ferritin immunoreactivity in the autopsied brain. Legal Med 5:S360-366). Because iron induces oxidative damage to tissues, resulting in age-related diseases, such as Alzheimer's disease, compounds that reduce iron overload and hyperferritinemia have been proposed as therapeutic targets for aging-associated diseases (Bartzokis G, Tishler T A, Lu P H, Villablanca P, Altshuler L L, Carter M et al. (2007) Brain ferritin iron may influence age- and gender-related risks of neurodegeneration. Neurobiol Aging 28:414-423).

Aging skin and poor wound healing can negatively impact quality of life. Aging skin has changes to structure and function that impairs its integrity and ability to heal (Farage M A, Miller K W, Elsner P, Maibach H I (2013) Characteristics of the aging skin. Adv Wound Care 2: doi: 10.1089). Compounds that prevent or correct intrinsic changes with age that influence skin integrity and repair can aid in improving the quality of life.

With respect to aging and pain, over half of people over 65 years old report to have bothersome pain, most of which have pain in multiple sites and have at least two chronic medical conditions such as arthritis, cardiometabolic diseases, and obesity (Patel K V et al. (2013) Prevalence and impact of pain among older adults in the United States: Findings from the 2011 National Health and Aging Trends Study. Pain 154:2649-2657). As such, anti-inflammatory agents, analgesics, and compounds that attenuate cardiometabolic diseases may aid in alleviating pain, including pain associated with aging. As an example, compounds that reduce prostaglandin E2 (PGE2) can aid in reducing inflammation, pain, and fever, including pain caused by osteoarthritis (Lee A S et al. (2013) A current review of molecular mechanisms regarding osteoarthritis and pain. Gene 527:440-447). Other compounds that reduce inflammation associated with autoimmune diseases, such as rheumatoid arthritis, can attenuate joint inflammation and pain. As an example, interleukin-17A (IL-17a) is an important contributor to autoimmune diseases, including multiple sclerosis and rheumatoid arthritis, and a compound that lowers IL-17A may help to alleviate pain from rheumatoid arthritis (Iwakura Y et al. (2008) The roles of IL-17A inflammatory immune responses and host defense against pathogens. Immunol Rev 226:). Further, compounds that reduce chronic systemic inflammation associated with aging and cardiometabolic diseases may attenuate these diseases and their associated pain.

With respect to aging and allergies, allergies are one of the fastest growing health problems in people aged over 15 years, and 5% to 10% of allergies are affecting elderly people (Martinis M D et al. (2017) Allergy and aging: an old/new emerging health issue. Aging and Dis 8:162-175). Elderly can be at higher risk of allergies due to physiological changes with aging, concurrent diseases, polydrug therapy, and compromised systems, including the dermal, gastrointestinal, and respiratory systems. Interleukin-17A (IL-17a) is an important contributor to allergic responses, including systemic and dermal hypersensitivities and allergic airway inflammation (Iwakura Y et al. (2008) The roles of IL-17A inflammatory immune responses and host defense against pathogens. Immunol Rev 226). As such, compounds that reduce allergy responses, including reduction of IL-17A, can improve the quality of life for people, including the elderly.

With respect to aging and sleep disorders, sleep problems have been reported to affect up to 40% of the elderly population (Vitiello M V (1997) Sleep disorders and aging: understanding the causes. J Gerontol 52A:M189-M191). This high prevalence is attributed, in part, to physiological changes with aging, as well as the presence of chronic disease. Control of chronic conditions, such as pain, have been demonstrated to improve the quality of sleep and resolve insomnia (Monjan A and Foley D (1996) Incidence of chronic insomnia associated with medical and psychosocial factors: an epidemiologic study among older persons. Sleep Res 25:108). Despite growing concern over the use of benzodiazapines and other sedatives, especially among the elderly, people over 60 years old are more likely to receive sedative prescriptions compare to people aged 40 to 59 (Baum C et al. (1986) Drug utilization in the U.S.—1985: Seventh annual review. Rockville, Md.: Food and Drug Administration, Center for Drugs and Biologies). As such, use of natural compounds that may help alleviate underlying medical conditions impacting sleep can be used as a first line means of improving the quality of sleep and life. As a potential alternative to sedatives, PGE2 inhibitors, which can be used to reduce inflammation, pain and fevers, may also aid in stemming PGE2's role in stimulating the wake centers near the posterior hypothalamus (Hayaishi O (1991) Molecular mechanisms of sleep-wake regulation: roles of prostaglandins D2 and E2. FASEB J 5:2575-2581).

With respect to aging and gastrointestinal and/or digestive disorders, aging impacts normal digestion, resulting in disorders associated with sensation, inflammation, poor swallowing, imbalanced microbiota, malabsorption, and malnutrition (Shamburek R D and Farrar J T (1990) Disorders of the digestive system in the elderly. New Engl J Med 322:438-443). People older than 65 years represent 25% of all inflammatory bowel disease hospitalizations, and older age was a significant risk factor for increased mortality and more severe gastrointestinal disease compared to younger patients (Ananthakrishnan A N et al. (2009) Inflammatory bowel disease in the elderly is associated with worse outcomes: a national study of hospitalizations. Inflamm Bowel Dis 15: 182-289). Compounds that lower gastrointestinal inflammation, restore a balanced microbiota and/or improve proper absorption of nutrients may aid in alleviating digestive disorders. Interleukin-17A (IL-17A) is a contributor to chronic intestinal inflammation, and lowering IL-17A secretion may aid in alleviating digestive disorders (Coccia M et al. (2012) IL-1β mediates chronic intestinal inflammation by promoting the accumulation of IL-17A secreting innate lymphoid cells and CD4+ Th17 cells. J Exp Med 209:1595).

With respect to aging and skin conditions and/or wound healing, advanced age can impair wound healing (Sgnoc R and Gruber J (2013) Age-related aspects of cutaneous wound healing: a mini review. Gerontol 59:159-164). This impairment can be due to chronic inflammatory conditions present with age, including increases in cytokines, such as IL-6, and chemokines, such as CXCL8 (also called interleukin 8 (IL-8)), which is elevated in psoriasis. There are some differences present in fetal and elderly wound healing, however, that may be beneficial. As an example, fetal and elderly wounds heal with no to little scarring, respectively. This scarless repair may be due to lower levels of decorin and IL-8, an extracellular matrix proteoglycan and chemokine, respectively, observed in fetal tissue (Beanes S R et al. (2001) Down-regulation of decorin, a transforming growth factor-beta modulator, is associated with scarless fetal wound healing. J Pediatr Surg 36:1666-71; Liechty K W (1997) Diminished interleukin-8 (IL-8) production in the fetal wound healing response. J Surg Res 77:80-84). As such, lower levels of IL-6, IL-8, and decorin may help stem inflammation associated with chronic, non-healing wounds and enable this healing to occur with minimal scarring.

It is an object of certain of the embodiments to provide a method for detecting protective factors for and risk factors against conditions provided herein, including but not limited to asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, and other related conditions in mammal subjects, such as companion animals and humans. An object of certain of the embodiments is to provide a method for treating conditions including but not limited to aging-associated conditions that impact quality of life or longevity in mammal subjects, such as companion animals and humans. An object of certain of the embodiments is to provide a method for detecting conditions including but not limited to aging-associated conditions that impact quality of life or longevity in mammal subjects, such as companion animals and humans. It is an object of certain of the embodiments to provide a method for increasing the serum, plasma, or erythrocyte membrane level of one or more small molecule metabolites or small molecule metabolite derivatives, including but not limited to biochemicals listed in Table 1, for example, an amino acid, such as 2-methylserine, and/or certain lipids, such as 10-undecanoate, in mammal subjects, such as companion animals and humans. An object of certain of the embodiments is to provide a small molecule metabolite supplement or prescription therapeutic for treating or preventing conditions including but not limited to those associated with aging and conditions that impact quality of life or longevity. An object of certain of the embodiments is to provide a method for detecting and/or treating aging-associated conditions that impact quality of life or longevity in mammal subjects, such as companion animals and humans, that is easy to accomplish in a cost-effective manner.

An object of certain of the embodiments is to employ the compositions of the embodiments in the prophylaxis, amelioration, and/or treatment of selected diseases and conditions. These diseases and conditions include but are not limited to oral indications, e.g., cavities (tooth decay); gum disease (gingivitis); periodontitis; sensitive teeth; oral cancer; burning mouth syndrome; ulcers, sores, or tender areas in the mouth; bleeding or swollen gums after brushing or flossing; chronic bad breath; sensitivity to hot and cold temperatures or beverages; pain or toothache; loose teeth; receding gums; pain with chewing or biting; swelling of the face and cheek; clicking of the jaw; frequent dry mouth; and stomatitis. These diseases and conditions include but are not limited to skin conditions, e.g., acne, cold sore, blister, hives, actinic keratosis, rosacea, carbuncle, exzcema, psoriasis, cellulitis, measles, basal cell carsinoma, squamous cell carcinoma, melanoma, lupus, contact dermatitis, vitiligo, wart, chickenpox, seborrheic eczema, keratosis pilaris, ringworm, melasma, impetigo, diaper rash, rashes from bacterial or fungal infections, rashes from allergic reactions, fifth disease, and vasculitis. These diseases and conditions include but are not limited to pain, e.g., chronic pain, nerve pain, psychogenic pain, musculoskeletal pain, chronic muscle pain, abdominal pain, joint pain, central pain syndrome, complex regional pain syndrome, diabetes related nerve pain (neuropathy), shingles pain (postherpetic neuralgia), and trigeminal neuralgia. These diseases and conditions include but are not limited to allergies, e.g., food allergies, seasonal allergies (e.g., hay fever, pollen), allergies to animal products (e.g., pet dander, dust mites, cockroaches), drugs such as penicillin and sulfa, foods (e.g., wheat, nuts, milk, shellfish, egg, legumes), insect stings (e.g., bees, wasps, mosquitoes), mold, plants (e.g., pollens from grass, weeds, trees; resins from plants such as poison ivy and poison oak), miscellaneous allergies (e.g., latex, nickel), contact dermatitis, rashes, eczema, sore throat, hives, swollen eyes, itching, burning, itchy eyes, watery eyes, runny nose, coughing, asthma, and airway inflammation. These diseases and conditions include but are not limited to sleep conditions, e.g., obstructive sleep apnea, central sleep apnea, insomnia, hypersomnia (daytime sleepiness), parasomnias, REM sleep behavior disorder, circadian rhythm sleep disorders, non-24-hour sleep-wake disorder, periodic limb movement disorder, shift work sleep disorder, and narcolepsy. These diseases and conditions include but are not limited to digestive and/or gastroinestinal conditions or disorders, e.g., diarrhea, constipation, acid reflux, heartburn, vomiting, gastroesophageal reflux disease, gallstones, celiac disease, Crohn's disease, ulcerative colitis, irritable bowel syndrome, hemorrhoids, diverticulitis, diverticulosis, anal fissure, peptic ulcers, gastroparesis, and nausea.

An object of certain of the embodiments is to provide a method for modulating markers of aging-associated conditions that impact quality of life or longevity in mammal subjects, such as companion animals and humans. An object of certain of the embodiments is to provide a method for detecting aging-associated conditions that impact quality of life or longevity in mammal subjects, such as companion animals and humans. An object of certain of the embodiments is to provide a method for treatment of aging-associated conditions that impact quality of life or longevity in mammal subjects, such as companion animals and humans. An object of certain of the embodiments is to provide a method for prophylaxis of aging-associated conditions that impact quality of life or longevity in mammal subjects, such as companion animals and humans. An object of certain of the embodiments is to provide a method for prophylaxis of a condition provided herein including aging-associated conditions that impact quality of life or longevity, including asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, and other related conditions, in mammal subjects, such as companion animals and humans.

An object of certain of the embodiments is to provide a method for increasing a small molecule metabolite in the sera, plasma, or erythrocyte membranes of mammal subjects, such as companion animals and humans. An object of certain of the embodiments is to provide a method for detecting or treating aging-associated conditions that impact quality of life or longevity in mammal subjects, such as companion animals and humans. An object of certain of the embodiments is to provide a small molecule metabolite substantially free from other small molecule metabolites in mammal subjects, such as companion animals and humans.

It is an object of certain of the embodiments is to provide a method for detecting and treating aging-associated conditions that impact quality of life or longevity in mammal subjects, such as companion animals and humans. An object of certain of the embodiments is to provide a small molecule metabolite, such as a biochemical listed in Table 1, for treating aging-associated conditions that impact quality of life or longevity in mammal subjects, such as companion animals and humans. An object of certain of the embodiments is to provide a method for prophylaxis of aging-associated conditions that impact quality of life or longevity in mammal subjects, such as companion animals and humans. An object of certain of the embodiments is to provide a method for detecting or treating aging-associated conditions that impact quality of life or longevity in mammal subjects, such as companion animals and humans. An object of certain of the embodiments is to provide a small molecule metabolite as described herein to supplement for treating conditions that impact quality of life or longevity in mammal subjects, such as companion animals and humans.

An object of certain of the embodiments is to provide a bioavailable form of small molecule metabolites to mammal subjects, such as companion animals and humans. An object of certain of the embodiments is to provide one or more small molecule metabolites described herein with one or more other small molecule biochemicals described herein to mammal subjects, such as companion animals and humans. An object of certain embodiments is to provide a method for increasing small molecule biochemicals described herein in the sera of mammal subjects, such as companion animals and humans. An object of certain of the embodiments is to provide a method for altering concentrations of a variety of small molecule metabolites as described herein in the sera, plasma, or erythrocyte membranes of mammal subjects, such as companion animals and humans.

Compositions including one or more of certain small molecule metabolites from contained herein, and associated methods for treatment of inflammation are provided. Compositions including one or more bioavailable metabolites contained herein are provided.

Compositions including one or more of certain small molecule metabolites from Table 1, and associated methods for treatment of anemia are provided. Compositions including one or more bioavailable metabolites contained herein are provided.

Compositions including one or more of certain small molecule metabolites from Table 1, and associated methods for treatment of hyperglycemia are provided. Compositions including one or more bioavailable metabolites contained herein are provided.

Compositions including one or more of certain small molecule metabolites from Table 1, and associated methods for treatment of dyslipidemia are provided. Compositions including one or more bioavailable metabolites contained herein are provided.

Compositions including one or more of certain small molecule metabolites contained herein, and associated methods for treatment of hyperinsulinemia are provided. Compositions including one or more bioavailable metabolites contained herein are provided.

Compositions including one or more of certain small molecule metabolites contained herein, and associated methods for treatment of liver disease are provided. Compositions including one or more bioavailable metabolites contained herein are provided.

Compositions including one or more of certain small molecule metabolites contained herein, and associated methods for treatment of iron overload are provided. Compositions including one or more bioavailable metabolites contained herein are provided.

Compositions including one or more of certain small molecule metabolites contained herein, and associated methods for treatment of impaired skin integrity, wound healing, scarring, pain, allergies, sleep disorders and problems, and gastrointestinal disorders and problems are provided. Compositions including one or more bioavailable metabolites contained herein are provided.

One or more than one of the aforementioned objects is provided by or achieved by the various compositions, methods, and uses as described herein.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “short-chain fatty acid” as used herein is a broad term, and is to be given its ordinary and customary meaning to a person of ordinary skill in the art (and is not to be limited to a special or customized meaning), and refers without limitation to a fatty acid with 2-6 carbon atoms

The term “medium-chain fatty acid” as used herein is a broad term, and is to be given its ordinary and customary meaning to a person of ordinary skill in the art (and is not to be limited to a special or customized meaning), and refers without limitation to a fatty acid with 7-12 carbon atoms.

The term “long-chain fatty acid” as used herein is a broad term, and is to be given its ordinary and customary meaning to a person of ordinary skill in the art (and is not to be limited to a special or customized meaning), and refers without limitation to a fatty acid with 13-22 carbon atoms.

The term “very long chain fatty acid” as used herein is a broad term, and is to be given its ordinary and customary meaning to a person of ordinary skill in the art (and is not to be limited to a special or customized meaning), and refers without limitation to a fatty acid with 23 or more carbon atoms.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, phosphates, triphosphates, and β-sulfenyl derivatives.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, small molecule metabolites, fatty alcohols, sterol and sterol derivatives, phospholipids, ceramides, sphingolipids, tocopherols, and carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

The term “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

The term “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

The term “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

The terms “treating,” “treatment,” “therapeutic,” or “therapy” as used herein are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

Any percentages, ratios or other quantities referred to herein are on a weight basis, unless otherwise indicated.

### Small Molecule Metabolites

Small molecule metabolites are low molecular weight (typically less than 900 daltons, but sometimes higher) and can include but are not limited to amino acids, peptides, xenobiotics, or lipids that can be identified and measured in the body and as provided herein. Small molecule metabolites can originate from ingestion of food or other oral products or produced endogenously. Small molecule metabolites are referred to and described using conventional nomenclature as is employed by one of skill in the art.

A small molecule metabolite may be referred to by various names, for example, 4-guanidinobutanoate may be referred to as gamma-Guanidinobutyric acid.

In some embodiments, the small molecule metabolite can be an amino acid, lipid, peptide or xenobiotic as provided herein. In further embodiments, one or more small molecule metabolites can include at least one amino acid, lipid, peptide or xenobiotic as provided herein.

Small molecule metabolites that are ideal candidates as both biomarkers and therapeutics are metabolites that are successfully detected in serum at high nanomolar or micromolar levels that have a low molecular weight (<900 daltons) and meet Lipinski's rule of five. All metabolites provided herein meet these criteria.

In some embodiments, a small molecule metabolite can be an amino acid, including but not limited to 4-guanidinobutanoate, 5-(galactosylhydroxyl)-L-lysine, N-acetylthreonine, S-adenosylhomocysteine (SAH), vanillylmandelate (VMA), or xanthosine. Derivatives can be synthesized by published methods.

In some embodiments, a small molecule metabolite can be a peptide, including but not limited to gamma-glutamylhistidine; a lipid, including but not limited to chenodeoxycholate, cholate, or myo-inositol; or a xenobiotic, including but not limited to hippurate. Derivatives can be synthesized by published methods.

In some embodiments, a derivative of a small molecule metabolite can be a β-sulfenyl derivative. It is thought that β-sulfenyl derivatives, such as an acid or ester, can be resistant to β-oxidation in the body. Derivatives can be synthesized by published methods.

In some embodiments, a small molecule metabolite is provided in a bioavailable form. The term “bioavailability” refers to the fraction of an administered dose of unchanged drug that reaches the systemic circulation, one of the principal pharmacokinetic properties of drugs. By definition, when a medication is administered intravenously, its bioavailability is 100%. As employed herein, the term “bioavailable” refers to a form of the small molecule metabolite that is successfully absorbed by the body when using methods of administration other than intravenous, for example, an oral therapeutic). In some embodiments, small molecule metabolite-based compositions may include adaptions that optimize absorption.

A pure or purified small molecule metabolite may exist in various physical states. For example, xanthosine exists as a solid that is stable at room temperature; this compound can be purchased in forms suitable for research purposes in small amounts from some commercial suppliers (for example, from Sigma-Aldrich Corp., of St. Louis, Mo.). Other small molecule metabolites, or stereoisomers, or solvates, or esters, or salts or other derivatives thereof, may exist as oils, solids, crystalline solids, or gases.

A small molecule metabolite or the pharmaceutically acceptable salts or derivatives thereof, may be provided in a purity (e.g., a percentage of the small molecule metabolite, or its pharmaceutically acceptable salts or derivatives, in a bulk form) of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure, wherein substantially pure may include, but not be limited to, a product with impurities at a level such that no physiological effect from the presence of the impurities is detectable. A mixture of small molecule metabolites, such as, for example, an amino acid and/or lipid, or pharmaceutically acceptable salts or derivatives thereof, may be present in a purity of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure. The small molecule metabolite, or a mixture thereof, or a pharmaceutically acceptable salt or derivative thereof, may be free from other small molecule metabolites. Without limitation, a small molecule metabolite as provided herein may be substantially free from other small molecule metabolites, singly or taken as a group; small molecule metabolites to exclude, for example, can include palmitic acid (C16:0) or stearic acid (C18:0). In some embodiments, a small molecule metabolite as provided herein may be substantially free from other species of lipids not included herein.

A small molecule metabolite, such as an amino acid, peptide, carbohydrate, cofactor, vitamins, xenobiotic, or lipid, or a pharmaceutically acceptable salt or derivative thereof, may be from any source. In some embodiments, a small molecule metabolite, or its pharmaceutically acceptable salts or derivatives, may be present in natural sources, may be isolated from natural sources, may be semi-synthetic, may be synthetic, or may be a mixture of one or more of these. The small molecule metabolite, or its pharmaceutically acceptable salts or derivatives, may be produced in a laboratory, may be produced in nature, may be produced by enzymatic processes, may be produced by wild microbes, may be produced by genetically modified microbes, may be isolated from animal tissues, may be produced by chemical synthesis, or may be produced by a plurality of these processes.

The small molecule metabolite may be derived from natural sources, e.g., plants, or can be synthesized by methods as are known in the art. In some embodiments, the small molecule metabolite may be contaminated with undesired components present in unrefined or unpurified natural products. In such situations, it can be desirable to remove undesired components, or to increase the concentration of desired components using known separation or purification techniques.

In any compound described, all tautomeric forms are also intended to be included. Without limitation, all tautomers of carboxylic groups are intended to be included.

In any compound described herein having one or more double bond(s) generating geometrical isomers that can be defined as E or Z, each double bond may independently be E or Z, or a mixture thereof.

Where compounds disclosed herein have unfilled valencies, then the valencies are to be filled with hydrogens or isotopes thereof, e.g., hydrogen-1 (protium) and hydrogen-2 (deuterium).

The small molecule metabolite, such as an amino acid, lipid, peptide, or xenobiotic, as described herein, includes crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The compounds described herein can be labeled isotopically. In some circumstances, substitution with isotopes such as deuterium may afford certain therapeutic advantages resulting from greater metabolic stability, such as, for example, increased in vivo half-life or reduced dosage requirements. Isotopic substitution may be beneficial in monitoring subject response to administration of a compound, for example, by providing opportunity for monitoring of the fate of an atom in a compound. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Compositions Including One or More Small Molecule Metabolites

Formulations including a small molecule metabolite, such as an amino acid, peptide, xenobiotic, or lipid, or a salt or derivative thereof, and at least one excipient are provided. It is generally preferred to administer the compounds of the embodiments in oral formulations; however, other routes of administration are also contemplated, such as topical. The formulations are suitable for use as consumer health and wellness products, including over the counter (OTC) products, as well as supplements and foodstuffs.

The compositions of the embodiments may be used in cosmetic, cosmeceutical and general skincare compositions or provided in pharmaceutical compositions, and can be employed in the promotion of healthy skin, skin regeneration and enhanced wound healing. Successful wound healing occurs when the tissue remodeling process alleviates the inflammatory response of the innate immune system and minimizes the scar forming process that occurs when fibrous tissue replaces normal skin after an injury. Less efficient or delayed wound healing often produces unsightly, irritating, and painful scars such as keloids or hypertrophic scars. The compositions can help treat or prevent dermatologic conditions such as skin dryness, dullness, loss of elasticity, lack of radiance, exaggerated lines and wrinkles, stretch marks, spider vessels, red blotchiness, smile lines, deep nasolabial fold lines, crow's feet, fine lines/wrinkles, vertical lines between the eyebrows, horizontal forehead lines, sagging thin/frail skin, skin redness, dullness, the appearance of photodamaged skin, the appearance of fine lines and wrinkles, hyperpigmentation, age spots, aged skin, and generally increasing the quality of skin.

The compositions of the embodiments can also be employed in the connection with mucous membranes, e.g., the lips and the vaginal mucosa. When applied to the vaginal mucosa, a vaginal applicator can be employed as are commercially available. Suitable applicators can be in a form of a pre-filled syringe, a tube attached to a prefilled squeezable reservoir, a prepackaged wand including a preselected amount of composition, or a universal vaginal applicator including perforations along its length for dispensing the composition through the perforations.

Certain of the compositions can contain further therapeutic agents, e.g., locally acting drugs such as antibacterial drugs, antiprotozoal drugs, antifungal drugs, antiviral drugs, spermicidal agents, prostaglandins, and steroids. Drugs suitable for delivery include bromocriptine, sildenafil, oxytocin, calcitonin, luteinizing hormone-releasing hormone and analogues, insulin, human growth hormone, oxybutynin, and steroids used in hormone replacement therapy or for contraception. Antifungal drugs include clotrimazole, econazole, miconazole, terbinafine, fluconazole, ketoconazole, and amphotericin. Antibiotics include amoxicillin, doxycycline, cephalexin, ciprofloxacin, clindamycin, metronidazole, azithromycin, sulfamethoxazole/trimethoprim, amoxicillin/clavulanate, and levofloxacin. Classes of antibiotics include penicillins, tetracyclines, cephalosporins, quinolones, lincomycins, macrolides, sulfonamides, glycopeptides, aminoglycosides, and carbapenems. Types of hormones include 5-alpha-reductase inhibitors, adrenal cortical steroids, corticotropin, glucocorticoids, mineralocorticoids, adrenal corticosteroid inhibitors, antiandrogens, antidiuretic hormones, antigonadotropic agents, antithyroid agents, aromatase inhibitors, calcitonin, estrogen receptor antagonists, gonadotropin-releasing hormone antagonists, growth hormone receptor blockers, growth hormones, insulin-like growth factor, parathyroid hormone and analogs, progesterone receptor modulators, prolactin inhibitors, selective estrogen receptor modulators, sex hormones, androgens and anabolic steroids, contraceptives, estrogens, gonadotropin releasing hormones, gonadotropins, progestins, sex hormone combinations, somatostatin and somatostatin analogs, synthetic ovulation stimulants, and thyroid drugs. Antiviral agents include adamantane antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors (NRTIs), protease inhibitors, and purine nucleosides. Drugs for treating skin conditions include acne drugs (isotretinoin), atopic dermatitis drugs (topical steroids), herpes zoster drugs (antivirals such as valacyclovir), hives (antihistamines like loratadine or fexofenadine, omalizumab), sunburn (lidocaine), contact dermatitis (antihistamines, topical steroids), diaper rash (zinc oxide), rosacea (metronidazole, doxycycline, azelaic acid, isotretinoin, beta blockers, estrogen), athlete's foot (antifungals), and basal cell carcinoma (imiquimod, fluorouracil, vismodegib).

The compositions of the embodiments include topical formulations containing at least one excipient. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for treatment of the skin. The composition can be formulated such that preservatives need not be employed.

To facilitate application, the composition may be provided as an ointment, an oil, a lotion, a paste, a powder, a gel, or a cream. The composition may also include additional ingredients such as a protective agent, an emollient, an astringent, a humectant, a sun screening agent, a sun tanning agent, a UV absorbing agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an additional antioxidant agent, a chemotherapeutic agent, an anti-histamine agent, a vitamin or vitamin complex, a hormone, an anti-dandruff agent, an anti-wrinkle agent, an anti-skin atrophy agent, a skin whitening agent, a cleansing agent, and combinations thereof. In a further embodiment, the composition may avoid animal or cellular-based materials to avoid skin irritation. The composition can be applied to the dermis, or to mucous membranes.

The compositions can be employed to promote healthy skin, skin regeneration, enhanced wound healing, and to treat skin conditions such as inflammation, redness, soreness, skin sensitivity, dry skin, bruising, and similar conditions. Suitable methods for objectively measuring improvement in skin redness and inflammation may include tristimulus colorimetry, narrow-band reflectance spectroscopy, diffuse reflectance spectroscopy, skin reflectance spectroscopy, and/or UV photography.

Some embodiments include administering the small molecule metabolite, such as an amino acid, peptide, carbohydrate, cofactor, vitamins, xenobiotic, or lipid, or a salt or derivative thereof in topical formulations; however, other routes of administration are also contemplated (e.g., mucosal, subdermal, oral, or the like) in addition to oral administration. Contemplated routes of administration include but are not limited to topical, mucosal, and subcutaneous. Suitable liquid forms include suspensions, emulsions, solutions, and the like. Unit dosage forms can also be provided, e.g., individual packets with a premeasured amount of the formulation, configured for administration to a body part on a predetermined schedule pre-procedure and post-procedure. Unit dosage forms configured for administration twice or three times a day are particularly preferred; however, in certain embodiments it can be desirable to configure the unit dosage form for administration once a day, four times a day, or more.

In some embodiments, the topical and other formulations typically comprise from about 0.001 wt. % or less to about 50 wt. % or more of active ingredient, such as the small molecule metabolite, such as an amino acid, peptide, carbohydrate, cofactor, vitamins, xenobiotic, or lipid, or a salt or derivative thereof, preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration can include transdermal patches, ointments, lotions, creams, gels, drops, sprays, liquids, aerosols, and powders. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

In some embodiments, the small molecule metabolite, such as an amino acid, peptide, carbohydrate, cofactor, vitamins, xenobiotic, or lipid, or a salt or derivative thereof, can be in combination therapy, or in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. Techniques for formulation and administration of the compounds described herein are known to those skilled in the art. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the small molecule metabolite, such as an amino acid, peptide, carbohydrate, cofactor, vitamins, xenobiotic, or lipid, or a salt or derivative thereof and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the small molecule metabolite, such as an amino acid, peptide, carbohydrate, cofactor, vitamins, xenobiotic, or lipid, or a salt or derivative thereof, into the skin. The pharmaceutical excipients used in the topical preparations of the compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the compositions as described herein may be dissolved or dispersed in a hydrophobic oil phase, and the oil phase may then be emulsified in an aqueous phase comprising water, alone or in combination with lower alcohols, glycerin, and/or glycols. Anhydrous formulations may also be employed; however, in certain embodiments it may be acceptable to provide water based compositions, or to permit a limited amount of water to be present.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

The small molecule metabolite, such as an amino acid, peptide, xenobiotic, or lipid contained herein, or a salt or derivative thereof, can be formulated as a liposome. The small molecule metabolite can be a component of the lipid portion of the liposome or can be encapsulated in the aqueous portion of the liposome. The small molecule metabolite, such as an amino acid, peptide, carbohydrate, cofactor, vitamins, xenobiotic, or lipid contained herein, or a salt or derivative thereof, can also be coformulated with a cyclodextrin. The cyclodextrin can be, for example, hydroxypropyl-β-cyclodextrin or a sulfobutylether cyclodextrin. Lecithin and other phospholipids may be used to prepare liposomes containing active ingredients as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the compositions as described herein.

The topical formulations may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others.

In certain embodiments systemic administration of the small molecule metabolite, such as an amino acid, peptide, carbohydrate, cofactor, vitamins, xenobiotic, or lipid, or a salt or derivative thereof, can be desirable. In such embodiments, the small molecule metabolite, such as an amino acid, peptide, carbohydrate, cofactor, vitamins, xenobiotic, or lipid, or a salt or derivative thereof are formulated into a composition suitable for oral administration, but other routes of administration are also contemplated.

The compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering a small molecule metabolite, such as an amino acid, peptide, carbohydrate, cofactor, vitamins, xenobiotic, or lipid, or a salt or derivative thereof, exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the small molecule metabolite, such as an amino acid, peptide, xenobiotic, or lipid, or a salt or derivative thereof may be combined as the active ingredient in intimate admixture with a pharmaceutical carrier according to conventional pharmaceutical compounding techniques. The small molecule metabolite, such as an amino acid, peptide, xenobiotic, or lipid, or a salt or derivative thereof can be added directly to, e.g., a gelatin capsule or a softgel capsule for consumption by the patient. In other embodiments, carriers can be employed. The carrier can take a wide variety of forms depending on the form of preparation desired for administration. Thus, the compositions provided herein can be presented as discrete units suitable for oral administration such as capsules, cachets or tablets each containing a predetermined amount of the active ingredient. Further, the compositions can be presented as an oil, a powder, as granules, as a solution, as a suspension in an aqueous liquid, as a non-aqueous liquid, as an oil-in-water emulsion, or as a water-in-oil liquid emulsion, similar to the topical formulations described elsewhere herein, but using components suitable for human consumption. In addition to the common dosage forms set out above, the compositions provided herein can also be administered by controlled release and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredients with liquid carriers or finely divided solid carriers or both. The product can then be conveniently shaped into the desired presentation.

A formulation may also be administered in a local rather than systemic manner, for example, via injection of the composition directly into a target area, e.g., in a depot or sustained release formulation. Furthermore, a targeted drug delivery system for the composition may be used, for example, in a liposome coated with a tissue specific antibody.

The compositions may contain the small molecule metabolite, such as an amino acid, peptide, xenobiotic, or lipid, or a salt or derivative thereof in an amount effective for the desired therapeutic effect. In some embodiments, the compositions are in a unit dosage form and comprise from about 0.1 mg or less to about 5000 mg or more of small molecule metabolite, such as an amino acid, peptide, carbohydrate, cofactor, vitamins, xenobiotic, or lipid, or a salt or derivative thereof per unit dosage form. In further embodiments, the compositions comprise from about 1 to about 500 mg per unit dosage form or from about 500 to 5000 mg per unit dosage form of small molecule metabolite, such as an amino acid, peptide, xenobiotic, or lipid, or a salt or derivative thereof. Such dosage forms may be solid, semisolid, liquid, an emulsion, or adapted for delivery via aerosol or the like for inhalation administration.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

The compositions provided herein can be prepared as solutions or suspensions of the small molecule metabolite, such as an amino acid, peptide, xenobiotic, or lipid, or a salt or derivative thereof in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Compositions provided herein suitable for injectable use include sterile aqueous solutions or dispersions. Furthermore, the compositions can be in the form of sterile powders for the extemporaneous preparation of such sterile injectable solutions or dispersions. The compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood or other bodily fluids of the intended recipient. Compositions containing a compound provided herein, or pharmaceutically acceptable salt or derivative thereof, can also be prepared in powder or liquid concentrate form for dilution.

Contemplated herein are compositions including the small molecule metabolite, such as an amino acid, peptide, xenobiotic, or lipid, or a salt or derivative thereof as described herein in combination with at least one additional active agent. The small molecule metabolite, such as an amino acid, peptide, xenobiotic, or lipid, or a salt or derivative thereof and the at least one additional active agent(s) may be present in a single formulation or in multiple formulations provided together, or may be unformulated. In some embodiments, the small molecule metabolite, such as an amino acid, peptide, xenobiotic, or lipid, or a salt or derivative thereof can be administered with one or more additional agents together in a single composition. For example, the small molecule metabolite, such as an amino acid, peptide, xenobiotic, or lipid, or a salt or derivative thereof can be administered in one composition, and at least one of the additional agents can be administered in a second composition. In a further embodiment, the small molecule metabolite, such as an amino acid, peptide, xenobiotic, or lipid, or a salt or derivative thereof and the at least one additional active agent(s) are co-packaged in a kit. For example, a drug manufacturer, a drug reseller, a physician, a compounding shop, or a pharmacist can provide a kit comprising the small molecule metabolite, such as an amino acid, peptide, xenobiotic, or lipid, or a salt or derivative thereof in combination with another product or component for delivery to a patient. Such additional components can include anti-infective agents, anti-inflammatory agents, anesthetics, or the like.

Some embodiments described herein relate to oral compositions of small molecule metabolite, such as an amino acid, peptide, xenobiotic, or lipid, or a salt or derivative thereof, which can include a therapeutically effective amount of the small molecule metabolite, such as an amino acid, peptide, xenobiotic, or lipid, or a salt or derivative thereof described herein and a pharmaceutically acceptable carrier, diluent, excipient or combination thereof. The compositions can include the small molecule metabolite, such as an amino acid, peptide, xenobiotic, or lipid, or a salt or derivative thereof in an amount for example, ≥1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition. In some embodiments, the pharmaceutical composition can include a plurality of small molecule metabolites, such as one or more of an amino acid, peptide, xenobiotic, and/or lipid described herein, or salts or derivatives thereof in, for example, ≥1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

### Foodstuffs

Foodstuffs and other comestibles including a small molecule metabolite, such as an amino acid, peptide, xenobiotic, or lipid described herein, or a salt or derivative thereof, are provided, wherein an amount of the small molecule metabolite in the foodstuff has been fortified (e.g., enriched or concentrated). A small molecule metabolite, such as an amino acid, peptide, xenobiotic, or lipid, provided herein may be added to foodstuffs for consumption by a subject. The small molecule metabolite, such as an amino acid, peptide, xenobiotic, or lipid described herein, may be integrated into one or more ingredients of a foodstuff. The small molecule metabolite, such as an amino acid, peptide, xenobiotic, or lipid described herein, may be prepared as an ingredient, or may be unprepared. The compound, or preparation including the compound, may be added prior to preparation, during preparation, or following preparation. Preparation may without limitation include cooking, mixing, flavoring, seasoning, blending, boiling, frying, baking, or other processes known in the art. Fortification is preferably at a level so as to provide a therapeutic daily dosage of the small molecule metabolite as described elsewhere herein; however, beneficial effects may also be obtained at amounts below such dosages.

A small molecule metabolite, such as an amino acid, peptide, xenobiotic, or lipid, or salt or derivative thereof, as provided herein may be present as a constituency in foodstuffs by operation of processes known in nature, for example, by altering the metabolic processes of a plant, animal, bacteria, or fungus. Genetic alteration of a plant, animal, bacteria, or fungus to increase the concentration of a small molecule metabolite, such as an amino acid, peptide, xenobiotic, or lipid as described herein, or a salt or derivative thereof, is contemplated. By way of example, the small molecule metabolite can be present in the foodstuff in a concentration of at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, or higher, for example, 1% to 2% or 3% or 4% or 5% or 6% or 7% or 8% or 9% or 10% or 20% or 30% or 40% or 50%. The small molecule metabolite, if naturally present in a foodstuff, can be present in an enriched amount above that which is naturally occurring for the foodstuff, e.g., a concentration of 10% or more above the average or highest naturally occurring observed concentration, e.g., 20% or 30% or 40% or 50% or 100% or 200% or 300% or 400% or 1000% or 2000% or 5000% or more above the average or highest naturally occurring observed concentration.

### Indications

Provided are compositions and methods for treating conditions that negatively impact longevity and the quality of aging, including but not limited to inflammation (including but not limited to inflammation of aging, obesity-associated inflammation, chronic low-lying inflammation, and autoimmune disorders (such as, for example Crohn disease, systemic lupus erythematosus, rheumatoid arthritis, psoriasis, type 1 diabetes, multiple sclerosis, and ulcerative colitis), hemolytic anemias (including but not limited to thalassemias, hereditary spherocytosis, hereditary elliptocytosis, glucose-6-phosphate dehydrogenase deficiency, pyruvate kinase deficiency, immune hemolytic anemia, alloimmune hemolytic anemia, drug-induced hemolytic anemia, mechanical hemolytic anemias, and paroxysmal nocturnal hemoglobinuria), anemia of chronic disease, anemia, aplastic anemias (including but not limited to congenital hypoplastic anemia, Diamond-Blackfan anemia and Fanconi anemia), iron deficiency anemia, anemias of abnormal RBC size (including but not limited to megaloblastic anemia and microcytic anemia), vitamin deficiency anemias (including but not limited to pernicious anemia) anemia of RBC mutation (including but not limited to thalassemia, sideroblastic anemia and sickle cell anemia), components of metabolic syndrome, including diabetes type II, obesity, pre-diabetes, glucose intolerance, gestational diabetes mellitus (GDM), impaired fasting glycemia (IFG), impaired adiponectin production, postprandial hyperglycemia, dyslipidemia, post prandial dyslipidemia, hyperlipidemia, hypertriglyceridemia, post hypertriglyceridemia, insulin resistance, polycystic ovary syndrome (PCOS), non-alcoholic fatty liver disease (NAFLD), non-alcoholic steatohepatitis (NASH), hypoinsulinemia, fatty liver disease, elevated glucose levels, elevated insulin levels, elevated LDL-cholesterol levels, elevated triglyceride levels, low HDL-cholesterol levels, and dysmetabolic iron overload syndrome (DIOS)), liver diseases, conditions with iron overload and/or hyperferritinemia (including but not limited to infection, neoplasm, chronic or acute inflammation, autoimmune diseases, DIOS and other iron overload and iron storage disorders, Still's disease, idiopathic arthritis, hemophagocytic lymphohistiocytosis, macrophage activation syndrome, liver conditions including NAFLD NASH, and hepatocellular carcinoma, anemia of chronic inflammation, and neurodegenerative diseases, including Alzheimer's disease and other forms of dementia), and delayed impaired skin integrity, delayed wound healing, and delayed scarring and impaired skin integrity, wound healing, and scarring (including delayed impaired skin integrity, wound healing, and scarring due to aging, obesity, chronic diseases, immunosuppression, nutritional status, burns, or vascular insufficiency), pain, allergies, sleep disorders and problems, and gastrointestinal disorders and problems.

Aging refers to a series of morphological and functional changes in an organism which take place over time. The term also refers to the deterioration of the biological functions after an organism has attained its maximum reproductive potential. It is thought that inflammation may be related to aging through mutation to mitochondrial DNA and other processes.

In some embodiments, the compositions and methods provided herein are indicated for treatment, prophylaxis, prevention or maintenance of aging-associated conditions, including hypercholesterolemia, obesity, thrombosis, fibrosis, impaired skin integrity, wound healing, scarring, hyperglobulinemia, hypersensitivity, cancer, pain, allergies, sleep disorders and problems, and gastrointestinal disorders and problems.

Without wishing to be limited by theory, it is thought that increasing small molecule metabolite levels in the serum, plasma, and cells to targeted concentrations may decrease aging-associated conditions.

In some embodiments, the methods provided herein increase levels of serum, plasma, or erythrocyte membrane small molecule metabolites.

In some embodiments, levels of serum, plasma, or erythrocyte membrane small molecule metabolites may increase following administration of one or more small molecule metabolites, or a salt or derivative thereof.

In some embodiments, the condition treated is anemia of chronic disease.

In some embodiments, the condition treated is autoimmune disease.

In some embodiments, the compositions and methods provided herein modulate a marker of aging-associated conditions that impair longevity or quality of life. In certain embodiments, the marker is serum, plasma, or red blood cell membrane small molecule metabolite percentage; serum, plasma, or red blood cell membrane concentration of a small molecule metabolite contained herein; or serum plasma, or red blood cell membrane total small molecule metabolite. In still further embodiments, the marker is serum or red blood cell membrane small molecule metabolite percentage, serum concentration of a small molecule metabolite, serum total small molecule metabolites.

In some embodiments, the methods provided herein include the step of measuring the concentration of a marker of inflammation. One of skill in the art will be able to perform suitable methods for such measurements, including but not limited to those described herein.

Provided herein are methods for treating including the step of administering a dose of a small molecule metabolite, such as a small molecule metabolite or a small molecule metabolite, at a predetermined interval, or at an interval left to the discretion of the subject.

In some embodiments, the compounds and methods provided herein may provide a threshold serum, plasma, or red blood cell membrane percentage of a small molecule metabolite relative to all serum, plasma, or red blood cell membrane small molecule metabolites, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of a small molecule metabolite, or red blood cell membrane concentration of a small molecule metabolite. For example, a serum or plasma small molecule metabolite or red blood cell membrane concentration of a small molecule metabolite may be increased by at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of a small molecule metabolite, or red blood cell membrane concentration of a small molecule metabolite may increase above a baseline value (e.g., pretreatment value in a patient being treated, or general value observed in a particular patient population) by at least about 0.01×10⁻⁴ M, at least about 0.05×10⁻⁴ M, at least about 0.1×10⁻⁴ M, at least about 0.2×10⁻⁴ M, at least about 0.3×10⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁴ M, at least about 2×10⁻⁴ M, or at least about 3×10⁻⁴ M.

In some embodiments, the compounds and methods provided herein may provide an increase in serum or plasma total small molecule metabolites, or red blood cell membrane total small molecule metabolites. For example, serum total small molecule metabolites, or red blood cell membrane total small molecule metabolites, may be increased above a baseline value (e.g., pretreatment value in a patient being treated, or general value observed in a particular patient population) by at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, the compounds and methods provided herein may provide an increase above a baseline value (e.g., pretreatment value in a patient being treated, or general value observed in a particular patient population) in a serum, plasma, or red blood cell membrane small molecule metabolites relative to all serum or red blood cell membrane small molecule metabolites, respectively. For example, a serum, plasma, or red blood cell membrane small molecule metabolite may be increased above a baseline value (e.g., pretreatment value in a patient being treated, or general value observed in a particular patient population) by at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.5%, at least about 4%, at least about 4.5%, at least about 5%, or more than 5%.

In some embodiments, the compounds and methods provided herein may provide a reduction in elevated erythrocyte sedimentation rate.

In some embodiments, the compounds and methods provided herein may provide a reduction in elevated alkaline phosphatase.

In some embodiments, the compounds and methods provided herein may provide a reduction in serum ferritin. For example, serum ferritin may be reduced below a baseline value (e.g., pretreatment value in a patient being treated, or general value observed in a particular patient population) by at least about 10 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 ng/ml, at least about 900 ng/ml, at least about 1000 ng/ml, at least about 1100 ng/ml, at least about 1200 ng/ml, at least about 1300 ng/ml, at least about 1400 ng/ml, at least about 1500 ng/ml, at least about 2000 ng/ml, at least about 2500 ng/ml, at least about 3000 ng/ml, at least about 3500 ng/ml, at least about 4000 ng/ml, at least about 4500 ng/ml, at least about 5000 ng/ml, at least about 6000 ng/ml, at least about 7000 ng/ml, at least about 8000 ng/ml, at least about 9000 ng/ml, at least about 10000 ng/ml, or more than 10000 ng/ml.

In some embodiments, the compounds and methods provided herein may provide a reduction in serum ferritin below a specified level. For example, serum ferritin may be reduced below about 20000 ng/ml, about 15000 ng/ml, about 12000 ng/ml, about 10000 ng/ml, about 8000 ng/ml, about 5000 ng/ml, about 2000 ng/ml, about 1000 ng/ml, or about 500 ng

In some embodiments, a small molecule metabolite is administered to maintain serum or plasma total percent of the small molecule metabolite, or all small molecule metabolites, above a predetermined threshold value. In variations of these embodiments, the small molecule metabolite is administered to maintain percent of the small molecule metabolite, above about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, or about 2.6%.

In some embodiments, the compounds and methods provided herein may provide a threshold serum, plasma, or red blood cell membrane percentage of a small molecule metabolite relative to all serum or red blood cell membrane small molecule metabolites, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of a small molecule metabolite, or red blood cell membrane concentration of a small molecule metabolite. For example, a serum small molecule metabolite or red blood cell membrane concentration of a small molecule metabolite may be increased by at least about 0.01 μg/ml, at least about 0.05 μg/ml, at least about 0.1 μg/ml, at least about 0.4 μg/ml, 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of a small molecule metabolite, or red blood cell membrane concentration of a small molecule metabolite may increase above a baseline value (e.g., pretreatment value in a patient being treated, or general value observed in a particular patient population) by at least about 0.001×10⁻⁴M, at least about 0.005×10⁻⁴ M, at least about 0.05×10⁻⁴ M, at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴M, at least about 0.6×10⁻⁴M, at least about 0.7×10⁻⁴M, at least about 0.8×10⁻⁴ M, at least about 0.9×10⁻⁴ M, at least about 1×10⁻⁴ M, at least about 2×10⁻⁴ M, or at least about 3×10⁻⁴ M.

In some embodiments, the compounds and methods provided herein may provide an increase in serum or plasma total small molecule metabolites, or red blood cell membrane total small molecule metabolites. For example, serum total small molecule metabolites, or red blood cell membrane total small molecule metabolites, may be increased above a baseline value (e.g., pretreatment value in a patient being treated, or general value observed in a particular patient population) by at least about 0.05 μg/ml, at least about 0.1 μg/ml, at least about 0.5 μg/ml, at least about 1 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, a composition or method provided herein may provide an increase in red blood cell count. For example, a red blood cell count level may be increased above a baseline value (e.g., pretreatment value in a patient being treated, or general value observed in a particular patient population) by at least about 0.1 cells/μL, at least about 0.2 cells/μL, at least about 0.3 cells/μL, at least about 0.4 cells/μL, at least about 0.5 cells/μL, at least about 0.6 cells/μL, at least about 0.7 cells/μL, at least about 0.8 cells/μL, at least about 0.9 cells/μL, at least about 1 cell/μL, at least about 1.2 cells/μL, at least about 1.4 cells/μL, at least about 1.6 cells/μL, or at least about 2 cells/μL.

### Combination Therapies

In some embodiments, the compounds disclosed herein, such as a small molecule metabolite, or a salt or derivative thereof, or a small molecule metabolite, or a salt or derivative thereof, or a pharmaceutical composition that includes a compound described herein, or a salt or derivative thereof, may be used in combination with one or more additional active agents. Examples of additional active agents that can be used in combination with a compound of an small molecule metabolite, or a salt or derivative thereof, or a composition that includes a compound of an small molecule metabolite, or a salt or derivative thereof, include, but are not limited to, agents currently used for treating conditions provided herein, and as otherwise known to medical science.

In some embodiments, a compound of a small molecule metabolite, or a salt or derivative thereof, or a composition that includes a compound of a small molecule metabolite, or a salt or derivative thereof, can be used with one, two, three or more additional active agents described herein. Such agents include, but are not limited to, a second small molecule metabolite, such as a small molecule metabolite, or a salt or derivative thereof.

In some embodiments, a compound of an small molecule metabolite, or a salt or derivative thereof, or a composition that includes a compound of a small molecule metabolite described herein, or a salt or derivative thereof, can be used (for example, administered or ingested) in combination with another agent or agents for treatment, prevention, maintenance, or prophylaxis of a condition provided herein including aging-associated conditions, including inflammation, anemia, hyperglycemia, dyslipidemia, hyperinsulinemia, liver disease, iron overload, impaired skin integrity, wound healing, scarring, pain, allergies, sleep disorders and problems, and gastrointestinal disorders and problems or for modulation of markers of the condition. In some embodiments, the condition can be inflammation (including but not limited to inflammation of aging, obesity-associated inflammation, chronic low-lying inflammation, and autoimmune disorders (such as, for example Crohn disease, systemic lupus erythematosus, rheumatoid arthritis, psoriasis, type 1 diabetes, multiple sclerosis, and ulcerative colitis), hemolytic anemias (including but not limited to thalassemias, hereditary spherocytosis, hereditary elliptocytosis, glucose-6-phosphate dehydrogenase deficiency, pyruvate kinase deficiency, immune hemolytic anemia, alloimmune hemolytic anemia, drug-induced hemolytic anemia, mechanical hemolytic anemias, and paroxysmal nocturnal hemoglobinuria), anemia of chronic disease, anemia, aplastic anemias (including but not limited to congenital hypoplastic anemia, Diamond-Blackfan anemia and Fanconi anemia), iron deficiency anemia, anemias of abnormal RBC size (including but not limited to megaloblastic anemia and microcytic anemia), vitamin deficiency anemias (including but not limited to pernicious anemia) anemia of RBC mutation (including but not limited to thalassemia, sideroblastic anemia and sickle cell anemia), components of metabolic syndrome, including diabetes type II, obesity, pre-diabetes, glucose intolerance, gestational diabetes mellitus (GDM), impaired fasting glycemia (IFG), impaired adiponectin production, postprandial hyperglycemia, dyslipidemia, post prandial dyslipidemia, hyperlipidemia, hypertriglyceridemia, post hypertriglyceridemia, insulin resistance, polycystic ovary syndrome (PCOS), non-alcoholic fatty liver disease (NAFLD), non-alcoholic steatohepatitis (NASH), hypoinsulinemia, fatty liver disease, elevated glucose levels, elevated insulin levels, elevated LDL-cholesterol levels, elevated triglyceride levels, low HDL-cholesterol levels, and dysmetabolic iron overload syndrome (DIOS)), liver diseases, conditions with iron overload and/or hyperferritinemia (including but not limited to infection, neoplasm, chronic or acute inflammation, autoimmune diseases, DIOS and other iron overload and iron storage disorders, Still's disease, idiopathic arthritis, hemophagocytic lymphohistiocytosis, macrophage activation syndrome, liver conditions including NAFLD NASH, and hepatocellular carcinoma, anemia of chronic inflammation, and neurodegenerative diseases, including Alzheimer's disease and other forms of dementia), and delayed impaired skin integrity, delayed wound healing, delayed scarring, and impaired skin integrity, wound healing, scarring (including delayed impaired skin integrity, wound healing, and scarring due to aging, obesity, chronic diseases, immunosuppression, nutritional status, burns, or vascular insufficiency), pain, allergies, sleep disorders and problems, and gastrointestinal disorders and problems. For example, a compound of a small molecule metabolite, such as a small molecule metabolite disclosed herein can be used in combination with one or more agents selected from iron chelators, albiglutide, aleglitazar, balaglitazone, canagliflozin, CJ-30001 (CJ Cheiljedang Corporation), CJ-30002 (CJ Cheiljedang Corporation), Diamyd® (glutamic acid decarboxylase (rhGAD65)), dulaglutide, exendin 4, gemigliptin, lixisenatide, lobeglitazone, shengke I (Tibet Pharmaceuticals), SK-0403 (Sanwa Kagaku Kenkyusho), teneligliptin, teplizumab, tofogliflozin, acarbose, alogliptin benzoate, chlorpropamide, Diab II (Biotech Holdings), exenatide, glibenclamide, gliclazide, glimepiride, glipizide, gliquidone, glisentide, glisolamide, HL-002 (HanAII Biopharma), insulin (human), insulin, insulin analogue (Eli Lilly®), insulin aspart, insulin detemir, insulin glargine, insulin lispro, Janumet®, linagliptin, liraglutide, metformin, miglitol, mitiglinide, nateglinide, Novo Mix 30® (Novo Nordisk®) pioglitazone, pramlintide, repaglinide, rosiglitazone maleate, saxagliptin, sitagliptin, Tresiba, tolazamide, tolbutamide, vildagliptin, voglibose, bezafibrate, diflunisal, cinnamic acid, carbutamide, glyburide (glibenclamide), glibomuride, glyhexamide, phenbutamide, and tolcyclamide or with one or more agents selected from a class of agents, where the classes include sulfonylureas, non-sulfonylurea secretagogues, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diguanides, diuretics, dopamine D1 receptor agonists, endopeptidase inhibitors, endothelin receptor antagonists, guanylate cyclase stimulants, phosphodiderivativease V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholderivativeyl derivative transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists lipase inhibitors, melanin-concentrating hormone receptor antagonists, nerve growth factor agonists, neuropeptide Y agonists, neuropeptide Y antagonists, SNRIs, protein tyrosine phosphatase inhibitors, serotonin 2C receptor agonists, or with other agents such as central nervous system agents that affect neurotransmitters or neural ion channels, including antidepressants (bupropion), noradrenalin reuptake inhibitors (GW320659), selective serotonin 2c receptor agonists, selective 5HT 2c receptor agonists, antiseizure agents (topiramate, zonisamide), dopamine antagonists, cannabinoid-1 receptor antagonists (CB-1 receptor antagonists) (rimonabant); leptin/insulin/central nervous system pathway agents, including leptin analogues, leptin transport and/or leptin receptor promoters, ciliary neurotrophic factor (Axokine), neuropeptide Y and agouti-related peptide antagonists, pro-opiomelanocortin and cocaine and amphetamine regulated transcript promoters, α-melanocyte-stimulating hormone analogues, melanocoritin-4 receptor agonists, and agents that affect insulin metabolism/activity, which include protein-tyrosine phosphatase-IB inhibitors, peroxisome proliferator activated receptor-.gamma. receptor antagonists, short-acting bromocriptine (ergoset), somatostatin agonists (octreotide), and adiponectin/Acrp30 (Famoxin or Small molecule metabolite Metabolic Oxidation Inducer); gastrointestinal-neural pathway agents, including those that increase cholecystokinin activity (CCK), PYY activity, NPY activity, and PP activity, increase glucagon-like peptide-1 activity (exendin 4, dipeptidyl peptidase IV inhibitors), and those that decrease ghrelin activity, as well as amylin analogues (pramlintide); agents that may increase resting metabolic rate (selective β-3 stimulators/agonist, uncoupling protein homologues, and thyroid receptor agonists); other more diverse agents, including melanin concentrating hormone antagonists, phytostanol analogues, functional oils, P57, amylase inhibitors, growth hormone fragments, synthetic analogues of dehydroepiandrosterone sulfate, antagonists of adipocyte 11B-hydroxysteroid dehydrogenase type 1 activity, corticotropin-releasing hormone agonists, inhibitors of small molecule metabolite synthesis (cerulenin and C75), carboxypeptidase inhibitors, indanone/indanols, aminosterols (trodusquemine/trodulamine), and other gastrointestinal lipase inhibitors (ATL962); amphetamines, such as dextroamphetamine; other sympathomimetic adrenergic agents, including phentermine, benzphetamine, phendimetrazine, mazindol, and diethylpropion; or with one or more agents selected from ecopipam; oxyntomodulin (OM); inhibitors of glucose-dependent insulinotropic polypeptide (GIP); gastrin-releasing peptide; neuromedin B; enterostatin; amfebutamone, SR-58611; CP-045598; AOD-0604; QC-BT16; rGLP-1; 1426 (HMR-1426); N-5984; ISIS-1 13715; solabegron; SR-147778; Org-34517; melanotan-II; cetilistat; c-2735; c-5093; c-2624; APD-356; radafaxine; fluasterone; GP-389255; 856464; S-2367; AVE-1625; T-71; oleoyl-estrone; peptide YY [3-36] intranasal; androgen receptor agonists; PYY 3-36; DOV-102677; tagatose; SLV-319; 1954 (Aventis Pharma AG); oxyntomodulin, Thiakis; bromocriptine, PLIVA; diabetes/hyperlipidemia therapy, Yissum; CKD-502; thyroid receptor beta agonists; beta-3 adrenoceptor agonist; CDK-A agonists; galanin antagonist; dopamine D1 D2 agonists; melanocortin modulators; verongamine; neuropeptide Y antagonists; melanin-concentrating hormone receptor antagonists; dual PPAR alpha/gamma agonists; CGEN-P-4; kinase inhibitors; human MCH receptor antagonists; GHS-R antagonists; ghrelin receptor agonists; DG70 inhibitors; cotinine; CRF-BP inhibitors; urocortin agonists; UCL-2000; impentamine; β-3 adrenergic receptor; pentapeptide MC4 agonists; trodusquemine; GT-2016; C-75; CPOP; MCH-1 receptor antagonists; RED-103004; aminosterols; orexin-1 antagonists; neuropeptide Y5 receptor antagonists; DRF-4158; PT-15; PTPase inhibitors; A37215; SA-0204; glycolipid metabolites; MC-4 agonist; produlestan; PTP-1B inhibitors; GT-2394; neuropeptide Y5 antagonists; melanocortin receptor modulators; MLN-4760; PPAR gamma/delta dual agonists; NPYSRA-972; 5-HT2C receptor agonist; neuropeptide Y5 receptor antagonists (phenyl urea analogs); AGRP/MC4 antagonists; neuropeptide Y5 antagonists (benzimidazole); glucocorticoid antagonists; MCHR1 antagonists; Acetyl-CoA carboxylase inhibitors; R-1496; HOB 1 modulators; NOX-B11; peptide YY 3-36 (eligen); 5-HT 1 modulators; pancreatic lipase inhibitors; GRC-1087; CB-1 antagonists; MCH-1 antagonists; LY-448100; bombesin BRS3 agonists; ghrelin antagonists; MC4 antagonists; stearoyl-CoA desaturase modulators; PPAR pan agonists; EP-01492; hormone-sensitive lipase inhibitors; small molecule metabolite-binding protein 4 inhibitors; thiolactone derivatives; protein tyrosine phosphatase IB inhibitors; MCH-1 antagonist; P-64; PPAR gamma ligands; melanin concentrating hormone antagonists; thiazole gastroprokinetics; PA-452; T-226296; A-331440; immunodrug vaccines; diabetes/obesity therapeutics (Bioagency, Biofrontera Discovery GmbH); P-7 (Genfit); DT-011 M; PTP1B inhibitor; anti-diabetic peptide conjugates; KATP agonists; obesity therapeutics (Lexicon); 5-HT2 agonists; MCH-1 receptor antagonists; GMAD-1/GMAD-2; STG-a-MD; angiogenesis inhibitors; G protein-coupled receptor agonists; nicotinic therapeutics (ChemGenex); anti-obesity agents (Abbott); melanin concentrating hormone; GW-594884A; MC-4R agonist; histamine H3 antagonists; orphan GPCR modulators; MITO-3108; NLC-002; HE-2300; IGF/BBP-2-13; 5-HT2C agonists; ML-22952; neuropeptide Y receptor antagonists; AZ-40140; anti-obesity therapy (Nisshin Flour); GNTI; melanocortin receptor modulators; alpha-amylase inhibitors; beta-3 adrenoceptor agonists; ob gene products (Eli Lilly & Co.); SWR-0342-SA; SWR-0335; SP-18904; oral insulin mimetics; obesity therapeutics (7TM Pharma); beta-hydroxysteroid dehydrogenase (HSD) inhibitors; QRX-431; E-6776; RI-450; melanocortin-4 antagonists; melanocortin 4 receptor agonists; obesity therapeutics (CuraGen); leptin mimetics; A-74498; second-generation leptin; NBI-103; CL-314698; CP-114271; beta-3 adrenoceptor agonists; NMI-8739; UCL-1283; BMS-192548; CP-94253; PD-160170; nicotinic agonist; LG-100754; SB-226552; LY-355124; CKD-711; L-751250; PPAR inhibitors; G-protein therapeutics; obesity therapy (Amylin Pharmaceuticals Inc.); BW-1229; monoclonal antibody (ObeSys/CAT); L-742791; (S)-sibutramine; MBU-23; YM-268; BTS-78050; tubby-like protein genes; genomics (eating disorders; Allelix/Lilly); MS-706; GI-264879A; GW-409890; FR-79620 analogs; obesity therapy (Hybrigenics SA); ICI-198157; ESP-A; 5-HT2C agonists; PD-170292; AIT-202; LG-100641; GI-181771; anti-obesity therapeutics (Genzyme); leptin modulator; GHRH mimetics; obesity therapy (Yamanouchi Pharmaceutical Co. Ltd.); SB-251023; CP-331684; BIBO-3304; cholesten-3-ones; LY-362884; BRL-48962; PY-1 antagonists; A-71378; .RTM.-didesmethylsibutramine; obesity therapeutics (Bristol-Myers Squibb Co.); obesity therapeutics (Ligand Pharmaceuticals Inc.); LY-226936; NPY antagonists; CCK-A agonists; FPL-14294; PD-145942; ZA-7114; CL-316243; SR-58878; R-1065; BDBP-3226; HP-228; talibegron; FR-165914; AZM-008; AZM-016; AZM-120; AZM-090; AZM-131; AZM-132; AZM-134; AZM-127; AZM-083; AZM-1 15; AZM-140; vomeropherin; BMS-187257; D-3800; gene discovery (Axys/Glaxo); BRL-26830A; SX-013; ERR modulators; adipsin; AC-253; A-71623; A-68552; BMS-210285; TAK-677; MPV-1743; obesity therapeutics (Modex); GI-248573; exopipam; SSR-125180; obesity therapeutics (Melacure Therapeutics AB); BRL-35135; SR-146131; P-57; CGP-71583A; RF-1051; BMS-196085; manifaxine; DMNJ (Korea Research Institute of Bioscience and Biotechnology); BVT-5182; LY-255582; SNX-024; galanin antagonists; neurokinin-3 antagonists; dexfenfluramine; mazindol; diethylpropion; phendimetrazine; benzphetamine; amfebutmone; sertraline; AOD-9604; ATL-062; BVT-933; GT389-255; SLV319; HE-2500; PEG-axokine; L-796568; and ABT-239; rimonabant, sibutramine, orlistat, PYY or an analog thereof, CB-1 antagonist, leptin, phentermine, and exendin analogs; GPR1 19 agonists (e.g., anandamide; AR-231, 453; MBX-2982; Oleoylethanolamide; PSN-365,963; PSN-632,408; palmitoylethanolamide); GPR120 agonists; GPR 40 agonists; and SGLT2 inhibitors.

Additionally, a small molecule metabolite or salt or derivative as provided herein can be used in combination with one or more agents selected from Altoprev (lovastatin), Crestor (rosuvastatin), Lescol (fluvastatin), Lipitor (atorvastatin), Livalo (pitavastatin), Pravachol (pravastatin), Zocor (simvastatin), an anti-platelet medication, a beta blocker, an ACE inhibitor, a calcium channel blocker, a diuretic, anticoagulants, aspirin, bile acid sequestrants, Ezetimibe, Fibrates, Glycoprotein IIb/IIIa Receptor Inhibitors, Niacin (Nicotinic Acid), Nitrates, Platelet Inhibitors, Thrombolytics, lisinopril oral, atenolol oral, Bystolic oral, Diovan oral, hydrochlorothiazide oral, metoprolol succinate oral, amlodipine oral, Norvasc oral, Toprol XL oral, Benicar oral, metoprolol tartrate oral, losartan oral, lisinopril-hydrochlorothiazide oral, clonidine HCl oral, Diovan HCT oral, Cozaar oral, propranolol oral, spironolactone oral, Azor oral, carvedilol oral, Coreg oral, Benicar HCT oral, Exforge oral, Avapro oral, Lotrel oral, verapamil oral, furosemide oral, Lasix oral, Hyzaar oral, Tekturna oral, enalapril maleate oral, Micardis oral, losartan-hydrochlorothiazide oral, ramipril oral, Lopressor oral, Altace oral, Micardis HCT oral, Avalide oral, diltiazem oral, triamterene-hydrochlorothiazide oral, labetalol oral, terazosin oral, amlodipine-benazepril oral, hydralazine oral, Atacand oral, benazepril oral, Tribenzor oral, triamterene oral, doxazosin oral, nifedipine oral, Ziac oral, Aldactone oral, Maxzide oral, Cartia XT oral, prazosin oral, Cardizem CD oral, Zestril oral, Dyazide oral, bisoprolol fumarate oral, Tenex oral, Tenormin oral, Coreg CR oral, Prinivil oral, valsartan oral, atenolol-chlorthalidone oral, Edarbyclor oral, benazepril-hydrochlorothiazide oral, ferrous sulfate oral, Ferrlecit intravenous, Feraheme intravenous, Feosol oral, Infed injection, Integra oral, Ferrex 150 Forte oral, Tandem Dual Action oral, Ferrex 150 oral, ferrous gluconate oral, Corvite 150 oral, Integra F oral, NovaFerrum oral, Iron (ferrous sulfate) oral, Vitron-C oral, Folic acid, corticosteroids, rituximab, IVIG, prednisone, methylprednisolone oral, Kenalog injection, Medrol (Pak) oral, Medrol oral, dexamethasone oral, Depo-Medrol injection, prednisolone oral, DexPak 13 Day oral, Solu-Medrol intravenous, hydrocortisone oral, Cortef oral, Deltasone oral, triamcinolone acetonide injection, cortisone oral, cholinesterase inhibitors such as Donepezil (Aricept), Rivastigmine (Exelon), and Galantamine (Razadyne), Memantine, Aricept, Namenda, Namenda XR, Razadyne ER, Alpha E, vitamin E, Hydergine, Namzaric, Dopamine Agonists such as pramipexole (Mirapex), ropinirole (Requip), rotigotine (Neupro patch) and apomorphine (Apokyn), Anticholinergics such as benztropine (Cogentin) and trihexyphenidyl, MAO-B Inhibitors such as (Eldepryl, Zelapar) and rasagiline (Azilect), COMT Inhibitors such as Entacapone (Comtan), Carbidopa/Levodopa (Sinemet®), amantadine, Tetrabenazine (Xenazine), haloperidol (Haldol), chlorpromazine, risperidone (Risperdal), quetiapine (Seroquel), olanzapine (Zyprexa), indomethacin, sulindac, etodolac, mefenamic acid, meclofenamic acid, meclofenamate sodium, flufenamic acid, tolmetin, ketorolac, diclofenac, diclofenac sodium, ibuprofen, naproxen, naproxen sodium, fenoprofen, ketoprofen, flurbiprofen, oxaprozin piroxicam, meloxicam, ampiroxicam, droxicam, lornoxicam, cinnoxicam, sudoxicam, and tenoxicam.

Additionally, a compound of a small molecule metabolite disclosed herein can be used in combination with one or more agents selected from iron dextran, iron sumalate, polysaccharide iron, ferrus fumarate, carbonyl iron, ferrous asparto glycinate, heme iron polypeptide can be sometimes indicated, ferrus bisglycinate as can be the administration of other medicaments such as androgen hormones, such as erythropoietin, folic acid, vitamin B12, vitamin C, succinic acid, niacin, pyridoxine, riboflavin, biotin, thiamine, calcium formate, Aminoxin, Anadrol-50, Chromagen Forte, Epoetin alfa, Epogen, Fe C Tab Plus, FeRiva, FeRivaFA, Ferocon, Ferotrin, Ferralet 90, Ferrex 28, Ferrogels Forte, FoliTab 500, Fumatinic, Hematogen Forte, Hemetab, Integra Plus, Irospan 42/6, Lenalidomide, Maxaron Forte, Myferon 150 Forte, MyKidz Iron, NovaFerrum, Oxymetholone, Procrit, Proferrin-Forte, Pyridoxine, Repliva 21/7, Revlimid, and Tricon.

### Dosing

As will be readily apparent to one skilled in the art, the useful in vivo dosage to be administered and the particular mode of administration will vary depending upon the age, weight, the severity of the condition, and mammalian species treated, the particular forms of the compounds employed, and the specific use for which these compounds are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of a condition provided herein including inflammation, anemia, hyperglycemia, dyslipidemia, hyperinsulinemia, liver disease, iron overload, impaired skin integrity, wound healing, scarring, pain, allergies, sleep disorders and problems, and gastrointestinal disorders and problems. In some embodiments, a therapeutically effective amount may be determined by reference to the modulation of a symptom of a condition provided herein. In still other embodiments, reference may be made to established guidelines for the conditions described herein, including, but not limited to, guidelines for the treatment of a condition provided herein including inflammation.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of a small molecule metabolite, such as an small molecule metabolite, or a salt or derivative thereof, or a mixture of a plurality of small molecule metabolites, or a salt or derivative thereof, from about 0.01 mg to about 10000 mg, from about 1 mg to about 5000 mg, from about 5 mg to about 2000 mg, from about 10 mg to about 1000 mg, or from about 50 mg to about 500 mg. A single dose may include a small molecule metabolite, or a salt or derivative thereof, in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher. The dosage may be a single one or a series of two or more given in the course of one or more days, as is appropriate for the individual subject. In some embodiments, the compounds will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, a small molecule metabolite, such as a small molecule metabolite, or a salt or derivative thereof, can be administered or ingested one time per day, two times per day, three times per day, or more.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

In some embodiments, the compounds and methods provided herein may be used in conjunction with devices and methods of using devices, for example, as provided in U.S. Pat. Nos. 7,651,845; 8,251,904; 8,251,904; 4,985,015; 8,827,957; 4,252,159; 5,318,521; 4,718,430; 9,713,600, 9,707,199, 9,687,461, 9,662,306, 9,561,206, U.S. Publ. No. 2011/0190702; U.S. Publ. No. 2017/0266144, U.S. Publ. No. 2016/0324814, U.S. Publ. No. 2016/0195559, U.S. Publ. No. 2016/0195558, U.S. Publ. No. 2016/0193172, 2 U.S. Publ. No. 016/0193171, U.S. Publ. No. 2016/0193170, WO 2016/111843, DE 2615061; and in conjunction with diagnostic devices, for example, as provided in U.S. Publ. No. 2012/0072236. The contents of each of the foregoing patent documents is incorporated herein by reference in its entirety.

### Diagnosis and Monitoring

Provided herein are methods for the diagnosis and monitoring of conditions provided herein including inflammation.

In some embodiments, the method of diagnosis or monitoring may comprise the step of measuring a percentage of a small molecule metabolite, such as an amino acid, peptide, xenobiotic, or lipid as described herein, in a bodily fluid. In some embodiments, the method of diagnosis or monitoring may comprise the step of measuring a marker of a condition provided herein including inflammation in a subject. In some embodiments, the method of diagnosis or monitoring may comprise the step of measuring a marker of anemia of chronic disease. In some embodiments, a correlation between one marker and another may prove instructive. In some embodiments, inflammation or a related condition may be diagnosed by reference to a threshold level of erythrocyte sedimentation rate, for example, or serum small molecule metabolite. In some embodiments, a condition provided herein including inflammation may be diagnosed by reference to a threshold level of a marker of the condition, for example, serum small molecule metabolite percentage, serum concentration of a small molecule metabolite, serum total small molecule metabolite, serum small molecule metabolite, or a ratio between two serum small molecule metabolites. For example, the threshold may be determined by reference to a symptom or marker of a condition provided herein including inflammation. For example, the condition can be metabolic syndrome.

The percentage of a small molecule metabolite, such as a small molecule metabolite, or a marker of a condition provided herein including inflammation, in a subject may be monitored by any means. Samples for analysis may be derived any fluid or tissue of the subject. For example, from serum, plasma, erythrocyte membranes, urine, and feces.

### EXAMPLES

### Example 1

Small molecule metabolites can serve as biomarkers, therapeutic targets, natural supplements, or therapeutics. The most promising metabolites are expected to 1) be detectable in blood, 2) have demonstrable differences between case and control populations, 3) have the ability to increase in concentration with interventions, and 4) have these increased levels correlated with clinical benefits. It was hypothesized that small molecule metabolite biomarkers, therapeutic targets, natural supplements, and therapeutics could be discovered by identifying metabolites that met at least three of four of the criteria above, using samples from bottlenose dolphin populations.

Bottlenose dolphins (Tursiops truncatus) are large-brained, long-lived mammals that develop aging-associated conditions similar to humans, including inflammation, anemia, hyperglycemia, dyslipidemia, hyperinsulinemia, liver disease, and iron overload (Venn-Watson S (2013) Blood-based indicators of insulin resistance and metabolic syndrome in bottlenose dolphins (Tursiops truncatus). Frontiers Endo 4:136, Venn-Watson S (2013) Associations of ceruloplasmin and haptoglobin with inflammation and glucose in bottlenose dolphins (Tursiops truncatus) J Comp Clin Path DOI: 10.1007/s00580-013-1738-0, Venn-Watson S (2012) Hemochromatosis and fatty change: building evidence for insulin resistance in bottlenose dolphins (Tursiops truncatus). J Zoo Wildlf Med 43:S35-S47, Venn-Watson S (2011) Physiology of aging among healthy, older bottlenose dolphins (Tursiops truncatus): comparisons with aging humans. J Comp Phys B 181:667-680, Venn-Watson S (2008) Clinical relevance of elevated transaminases in a bottlenose dolphin (Tursiops truncatus) population. J Wildlf Dis 44: 318-330). For over 60 years, the U.S. Navy has cared for a population of approximately 100 dolphins. While the average lifespan of dolphins in the wild is 20 years, Navy dolphins on average live 32 years, more than fifty percent longer than wild dolphins. Approximately one-third of Navy dolphins are between 30 to 50 years old. Due in part to these populations' age differences, Navy dolphins have higher mean glucose, cholesterol, triglycerides, GGT, insulin, iron, and ferritin levels compared to wild dolphins living in Sarasota Bay, Fla.

In addition to differences in mean age, Navy and wild dolphins ingest different fish-based diets. Historically, Navy dolphins have been fed a diet of small, low-fat species, such as capelin and squid. Wild dolphins in Sarasota Bay ingest a large variety of fish, including larger, high-fat species, such as mullet and pinfish. It has been previously demonstrated that feeding Navy dolphins a modified diet resembling that of wild dolphins resulted in improved clinical indices of aging-associated conditions, including lowered ferritin and normalized glucose, insulin, and triglycerides.

Routine, fasted serum samples collected throughout 26 Navy dolphins' lifespans and archived at −80° C. were submitted for global metabolomics and complex lipid profiling. The sample set targeted one sample for each dolphin's year of life, resulting in 355 samples for the study with paired clinical pathology data. In addition, single serum samples from 38 wild dolphins living in Sarasota Bay, Fla. were included in the sample set.

Navy dolphins were categorized based on the presence or absence of the following criteria during the most recent year of life (if alive) or during the year preceding death (if dead): at least 50% of routine and fasted blood samples with elevated glucose (greater than 95 mg/dl), elevated cholesterol (greater than 250 mg/dl), elevated triglycerides (greater than 137 mg/dl), elevated erythrocyte sedimentation rate (greater than 19 mm/hr), elevated liver enzymes (ALT, AST, or GGT levels greater than 41, 255, and 31 U/L respectively), or elevated iron (greater than 300 μg/dl). Animals were categorized as Early Cases (presence of two or three of the criteria), Advanced Cases (presence of more than three criteria), and Controls (presence of one or less of the criteria). Wild dolphins were considered a second Control group.

Serum samples were analyzed for small molecules using global metabolomics and complex lipid profiling. Briefly, serum extracts were prepared and analyzed using three methods: ultrahigh performance liquid chromatography-tandem mass spectroscopy (UPLC-MS/MS) with positive ion mode electrospray ionization (ESI), UPLC-MS/MS with negative ion mode ESI, and hydrophilic interaction liquid chromatography (UPLC-MS/MS).

The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

Raw data were extracted, peak-identified and QC processed using proprietary hardware and software. Compounds were identified by comparison to library entries of purified standards or recurrent unknown entities. Biochemical identifications were based on three criteria: retention index within a narrow RI window of the proposed identification, accurate mass match to the library+/−10 ppm, and the MS/MS forward and reverse scores between the experimental data and authentic standards. Peaks were quantified using area-under-the-curve. A data normalization step was performed to correct variation resulting from instrument inter-day tuning differences.

Serum samples were also analyzed for complex lipids. Briefly, lipids were extracted from samples in methanol:dichloromethane in the presence of internal standards. The extracts were concentrated under nitrogen and reconstituted in 0.25 mL of 10 mM ammonium acetate dichloromethane:methanol (50:50). The extracts were transferred to inserts and placed in vials for infusion-MS analysis, performed on a Shimazdu LC with nano PEEK tubing and the Sciex Selexlon-5500 QTRAP. The samples were analyzed via both positive and negative mode electrospray. The 5500 QTRAP scan was performed in MRM mode with the total of more than 1,100 MRMs. Individual lipid species were quantified by taking the peak area ratios of target compounds and their assigned internal standards, then multiplying by the concentration of internal standard added to the sample. Lipid class concentrations were calculated from the sum of all molecular species within a class, and small molecule metabolite compositions were determined by calculating the proportion of each class comprised by individual small molecule metabolites.

Several statistical models were used to identify targeted biochemicals. Welch's two-sample t-Tests were performed to compare metabolite quantities between Navy dolphins and wild dolphins. Table 1 summarizes small molecule metabolites that were significantly higher in wild dolphins compared to Navy dolphins.

TABLE 1

| | Ratio of serum-based levels in wild versus |
|---|---|
| Small molecule metabolites | Navy dolphins (P-value <0.05) |
| 4-guanidinobutanoate | 6.38 |
| chenodeoxycholate | 9.75 |
| cholate | 4.51 |
| hippurate | 1.99 |
| myo-inositol | 1.53 |
| N-acetylthreonine | 1.93 |
| S-adenosylhomocysteine | 1.30 |
| (SAH) | |
| xanthosine | 2.01 |

Linear correlations were used to test for associations between specific biochemicals and clinical pathology indices, specifically glucose, neutrophils, cholesterol, triglycerides, GGT, lymphocytes, iron, and red blood cells. Table 2 summarizes small molecule metabolites that negatively correlated with glucose, neutrophils, triglycerides, GGT, lymphocytes, and/or iron; and/or positively correlated with red blood cells.

TABLE 2

| Small | | | | | | | | | | | | |
|---|---|---|---|---|---|---|---|---|---|---|---|---|
| molecule | | | | | | | | | | | | |
| metabolites | | | | | | | | | | | | |
| in serum | | | | | | | | | | | | |
| and | | | | | | | | | | | | |
| correlations | | | | | | | | | | | | |
| with | | | | | Dyslipidemia | | | | | | | |
| clinical | Anemia | | Inflammation | | (Triglyc- | | Liver Disease | | Iron Overload | | Hyperglycemia | |
| indices | (RBCs) | | (Neutrophils) | | erides) | | (GGT) | | (Iron) | | (Glucose) | |
| in | P | | P | | P | | P | | P | | P | |
| dolphins | value | R² | value | R² | value | R² | value | R² | value | R² | value | R² |
| 4-guanidino- | — | — | 0.023 | −0.12 | — | — | — | — | — | — | 0.037 | −0.11 |
| butanoate | | | | | | | | | | | | |
| 5-(galacto- | — | — | 0.024 | −0.12 | — | — | — | — | <0.001 | +0.25 | — | — |
| sylhydroxy)- | | | | | | | | | | | | |
| L-lysine | | | | | | | | | | | | |
| chenodeoxy- | — | — | 0.03 | −0.12 | — | — | — | — | — | — | 0.005 | −0.15 |
| cholate | | | | | | | | | | | | |
| cholate | — | — | 0.01 | −0.13 | — | — | — | — | — | — | — | — |
| gamma- | 0.004 | +0.15 | 0.0002 | −0.20 | — | — | 0.030 | −0.11 | — | — | — | — |
| glutamyl- | | | | | | | | | | | | |
| histidine | | | | | | | | | | | | |
| hippurate | 0.005 | +0.15 | 0.008 | −0.14 | 0.0007 | −0.18 | <0.001 | −0.30 | | | 0.0007 | −0.18 |
| myo-inositol | — | — | — | — | — | — | — | — | — | — | 0.0002 | +0.19 |
| N-acetyl- | — | — | 0.025 | −0.12 | — | — | — | — | — | — | 0.004 | −0.15 |
| threonine | | | | | | | | | | | | |
| S-adenosyl- | 0.015 | +0.13 | — | — | — | — | — | — | 0.047 | +0.10 | 0.007 | −0.14 |
| homo- | | | | | | | | | | | | |
| cysteine | | | | | | | | | | | | |
| (SAH) | | | | | | | | | | | | |
| vanillyl- | — | — | <0.0001 | −0.24 | <0.0001 | −0.31 | <0.0001 | −0.48 | — | — | — | — |
| mandelate | | | | | | | | | | | | |
| (VMA) | | | | | | | | | | | | |
| xanthosine | — | — | 0.023 | −0.12 | — | — | — | — | — | — | 0.037 | −0.11 |

The relevance of the presence of age category to each biochemical was assessed using two-way ANOVA main effects considering disease category, age, and a mixed disease:age bin calculation. Table 3 summarizes significant (P value ≤0.05) effects of age, disease, and/or age+disease on small molecule metabolites in the animal study population.

TABLE 3

| | Two-Way ANOVA Main Effects | | |
|---|---|---|---|
| | (P value) | | |
| | | | Age + |
| | Age | Disease | Disease |
| Biochemical | Effect | Effect | Effect |
| 4-guanidinobutanoate | — | 0.006 | — |
| chenodeoxycholate | <0.0001 | <0.0001 | 0.046 |
| cholate | — | 0.021 | — |
| gamma-glutamylhistidine | 0.0007 | — | — |
| hippurate | <0.0001 | 0.031 | <0.0001 |
| myo-inositol | — | <0.0001 | — |
| N-acetylthreonine | — | — | 0.004 |
| S-adenosylhomocysteine (SAH) | 0.012 | — | — |
| vanillylmandelate (VMA) | <0.0001 | — | 0.007 |
| xanthosine | — | — | <0.0001 |

### Example 2

Bottlenose dolphins (Tursiops truncatus) are large-brained, long-lived mammals that develop aging-associated conditions similar to humans, including inflammation, anemia, hyperglycemia, dyslipidemia, hyperinsulinemia, liver disease, and iron overload. For over 60 years, the U.S. Navy has cared for a population of approximately 100 dolphins. While the average lifespan of dolphins in the wild is 20 years, Navy dolphins on average live 32 years, more than fifty percent longer than wild dolphins. Approximately one-third of Navy dolphins are between 30 to 50 years old. Due in part to these populations' age differences, Navy dolphins have higher mean glucose, cholesterol, triglycerides, GGT, insulin, iron, and ferritin levels compared to wild dolphins living in Sarasota Bay, Fla. Proposed risk factors for these conditions in MMP dolphins include advanced age and diet (see Venn-Watson S, Smith C R, Gomez F, Jensen E D (2011) Physiology of aging among healthy, older bottlenose dolphins (Tursiops truncatus): comparisons with aging humans. J Comp Phys B 181:667-680). It can be hypothesized that differences in dietary fish (and differences in certain small molecule metabolites associated with fish-based dietary changes) can be responsible for the risk of exacerbating conditions that negatively impact longevity and quality of life, including anemia, hypercholesterolemia, and hyperinsulinemia.

This study examined the impact on anemia by modifying serum metabolites through a modified fish diet in 20 MMP dolphins (“Modified Diet”). The dolphins lived in netted enclosures within San Diego Bay. The diets of the 20 modified diet dolphins were modified from a 75% capelin (plus 25% mix of squid, herring or mackerel) baseline diet to a diet consisting of 25% capelin, 50% mullet, and 25% mix of squid, herring, or mackerel while maintaining the same kilocalories. On blood collection days, modified diet dolphins were fed one-third of their daily diet in the morning after their routine overnight fast and 2 h postprandial, in-water, and trained blood samples were drawn (typically near 10:00 a.m.). An additional ten MMP dolphins (“Baseline Diet”) were maintained on the baseline capelin diet throughout the study period. There were no differences in age, sex, or body weight when comparing the two groups (Table 4).

Table 4 provides comparisons of blood-based clinical values among modified diet dolphins and baseline diet dolphins. *=p value ≤0.05.

TABLE 4

| | Month 0 | | Month 1 | | Month 3 | | Month 6 | |
|---|---|---|---|---|---|---|---|---|
| | Modified | Baseline | Modified | Baseline | Modified | Baseline | Modified | Baseline |
| | Diet | Diet | Diet | Diet | Diet | Diet | Diet | Diet |
| | (n = 20) | (n = 10) | (n = 20) | (n = 10) | (n = 20) | (n = 10) | (n = 20) | (n = 10) |
| Age (years) | 22 ±14 | 26 ± 10 | | | | | | |
| Body weight (lbs) | 389 ± 49 | 402 ± 45 | | | | | | |
| Female (%) | 45% | 40% | | | | | | |
| Insulin | 9.8 ± 6.1 | 9.4 ± 4.8 | 7.5 ± 04.0* | 14.8 ± 14.0 | 8.9 ± 4.4* | 16.3 ± 13.6 | 6.8 ± 4.6* | 10.3 ± 6.2 |
| Cholesterol | 183 ± 35 | 187 ± 23 | 160 ± 26* | 186 ± 24 | 167 ± 28* | 199 ± 33 | 160 ± 31* | 188 ± 2 8 |
| Hemoglobin | 13 ± 1 | 13 ± 1 | 14 ± 1 | 13 ± 1 | 15 ± 1* | 14 ± 1 | 15 ± 1* | 13 ± 1 |
| Hematocrit | 44 ± 5 | 44 ± 3 | 46 ± 3* | 44 ± 3 | 47 ± 2* | 45 ± 2 | 47 ± 2* | 44 ± 2 |
| RDW | 15 ± 2 | 15 ± 2 | 14 ± 1 | 15 ± 2 | 13 ± 1* | 15 ± 2 | 13 ± 1* | 16 ± 3 |
| RBC | 3.0 ± 0.3 | 3.0 ± 0.2 | 3.2 ± 0.2 | 3.0 ± 0.2 | 3.4 ± 0.2* | 3.2 ± 0.2 | 3.3 ± 0.3* | 3.1 ± 0.2 |
| NRBC | 1.8 ± 2.9 | 1.4 ± 2.5 | 0.5 ± 0.9* | 1.2 ± 1.5 | 0.2 ± 0.6* | 1.1 ± 1.0 | 0.2 ± 0.5 | 0.7 ± 1.3 |

Two-hour post-prandial samples were collected from modified diet dolphins and baseline diet dolphins at baseline (month 0) and at three time points following the switch to the modified diet: months 1, 3, and 6. Dolphins were assessed for changes in serum metabolites, cholesterol, insulin, and indices of anemia, including hematocrit, packed cell volume, red blood cell count, hemoglobin, and red blood cell distribution.

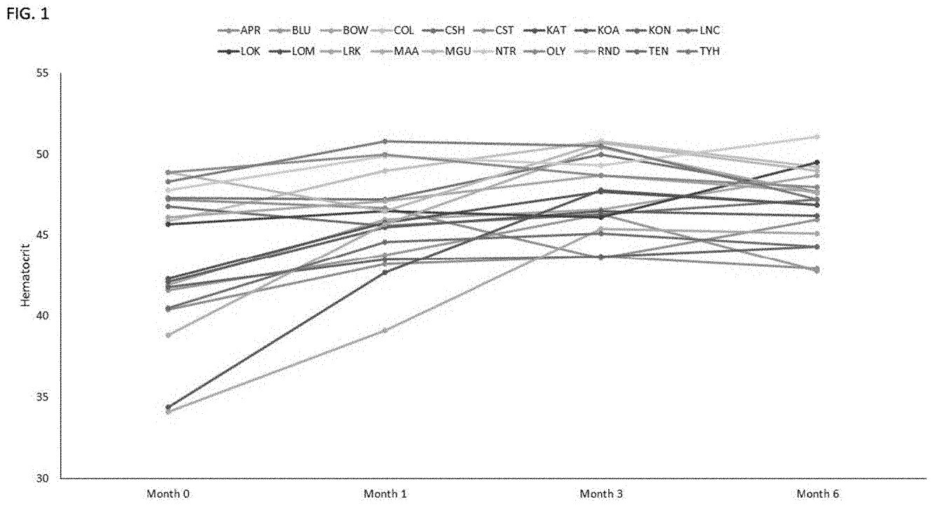

Changes in serum metabolites, as well as cholesterol, insulin, and indices of anemia, including hematocrit, packed cell volume, red blood cell count, hemoglobin, and red blood cell distribution, were compared in study dolphins during months 1, 3, and 6 and compared to month 0 using repeated Wilcoxon rank sum tests. Outcomes for clinical markers for modified diet dolphins and baseline diet dolphins are provided in Table 4. Cholesterol, insulin and all indicators of anemia improved in modified diet dolphins by Month 1 and through Month 6. No changes were identified in baseline diet dolphins. It is apparent in FIG. 1. that individual modified diet dolphins with low hematocrit had resolving anemia while on the modified diet.

Three small molecule metabolite concentrations in serum were successfully and significantly raised in this study (Table 5). Increases in serum metabolite concentrations ranged from 1.1 to 3.1-fold higher than baseline. Table 5 summarizes small molecule metabolites in serum that increased significantly among 1) dolphins on the modified diet (Month 1, Month 3, or Month 6) compared to the baseline control group, or 2) dolphins on the modified diet during later versus earlier months on the diet.

TABLE 5

| | Significant fold differences | |
|---|---|---|
| Small | in serum between Baseline | Significant fold differences |
| molecule | and Modified Diet groups | in serum between Months |
| metabolite | (earliest month on diet) | in Modified Diet group |
| 4-guanidino- | — | 1.38 (Month 6\|Month 1) |
| butanoate | | |
| chenodeoxy- | 2.97 (Month 1) | 3.11 (Month 3\|Month 0) |
| cholate | | |
| hippurate | — | 1.12 (Month 6\|Month 0) |

Demonstrated increased serum concentrations of specific small molecule metabolites in Group A dolphins on the modified diet correlated with lowered cholesterol, lowered insulin, and improved anemia (Table 6). Thus, these data support that raising serum concentrations of specific small molecule metabolites lowered cholesterol, lowered insulin, and resolved anemia. Table 6 summarizes four small molecule metabolites that significantly increased among modified diet dolphins and correlated with demonstrated clinical benefits, specifically lower cholesterol, lower insulin and/or raised red blood cells (RBCs).

TABLE 6

| | Associations between serum metabolite levels and aging-associated | | | | | |
|---|---|---|---|---|---|---|
| | disease biomarkers in dolphins | | | | | |
| | Red Blood Cells | | Cholesterol | | Insulin | |
| Small molecule metabolite | P value | R² | P value | R² | P value | R² |
| 4-guanidinobutanoate | — | — | — | — | 0.009 | −0.24 |
| chenodeoxycholate | <0.0001 | +0.36 | 0.010 | −0.24 | — | — |
| cholate | 0.011 | +0.23 | — | — | — | — |
| hippurate | 0.0002 | +0.33 | — | — | <0.0001 | −0.37 |

In summary, results from the dolphin study demonstrated that 1) serum-based metabolites can be altered by dietary interventions, 2) metabolite concentrations in the serum increased between 1.1 to 3-fold higher than baseline, pre-intervention levels, 3) increased serum concentrations of specific small molecule metabolites correlated with demonstrated clinical benefits, including lower cholesterol, lower insulin, and/or alleviated anemia.

### Sample Collection and Transport

Blood was collected into BD Vacutainer serum separator tubes (for serum fatty acid profiles). Serum separator tubes were centrifuged at 3000 rpm for 10 minutes within 30 to 60 minutes of collection and chilled during processing until shipment. Serum was transferred to cryovials and stored at −80° C. until shipment on dry ice via overnight courier to the reference laboratories.

### Sample Analysis

Red blood cell indices, including red blood cell count and hemoglobin, were performed by the Naval Medical Center San Diego. Betadine and alcohol swabs were used to clean the ventral aspect of the dolphin's fluke using the appropriate aseptic technique. Blood was then drawn with a 21 g×¾ in. winged infusion set with a luer adapter and vacutainer hub. A BD vacutainer blood tube with 7.2 mg EDTA was then applied to the luer adapter and blood was evacuated using the vacuum from the tubes. The blood was then shipped on ice packs to reference laboratories. A 4 ml EDTA vacutainer was shipped on an ice pack and analyzed by the Naval Medical Center of San Diego. The automated HCT, hemoglobin and red blood cell count were analyzed using the Sysmex XE-5000 (Sysmex Canada Inc., Mississauga, Ontario) per the manufacturer's protocol. For in-house packed cell volume, whole blood was taken from a 4 mL BD Vacutainer tube with 7.2 mg EDTA and used to fill a heparinized mylar wrapped 75 MM hematocrit tubes. Clay was packed into one end to prevent leakage during centrifugation. Once the hematocrit tube was sealed, it was placed into the Thermo IEC MICRO-MB centrifuge (Thermo Fisher Scientific, Waltham, Ma. 02451). The blood sample was spun at 11,500 rpm for 5 minutes. The hematocrit tube was then removed and placed into the micro-capillary reader to determine results.

Serum samples were analyzed for small molecules using global metabolomics and complex lipid profiling. Briefly, serum extracts were prepared and analyzed using three methods: ultrahigh performance liquid chromatography-tandem mass spectroscopy (UPLC-MS/MS) with positive ion mode electrospray ionization (ESI), UPLC-MS/MS with negative ion mode ESI, and hydrophilic interaction liquid chromatography (UPLC-MS/MS).

The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

Raw data were extracted, peak-identified and QC processed using proprietary hardware and software. Compounds were identified by comparison to library entries of purified standards or recurrent unknown entities. Biochemical identifications were based on three criteria: retention index within a narrow RI window of the proposed identification, accurate mass match to the library+/−10 ppm, and the MS/MS forward and reverse scores between the experimental data and authentic standards. Peaks were quantified using area-under-the-curve. A data normalization step was performed to correct variation resulting from instrument inter-day tuning differences.

Serum samples were also analyzed for complex lipids. Briefly, lipids were extracted from samples in methanol:dichloromethane in the presence of internal standards. The extracts were concentrated under nitrogen and reconstituted in 0.25 mL of 10 mM ammonium acetate dichloromethane:methanol (50:50). The extracts were transferred to inserts and placed in vials for infusion-MS analysis, performed on a Shimazdu LC with nano PEEK tubing and the Sciex Selexlon-5500 QTRAP. The samples were analyzed via both positive and negative mode electrospray. The 5500 QTRAP scan was performed in MRM mode with the total of more than 1,100 MRMs. Individual lipid species were quantified by taking the peak area ratios of target compounds and their assigned internal standards, then multiplying by the concentration of internal standard added to the sample. Lipid class concentrations were calculated from the sum of all molecular species within a class, and small molecule metabolite compositions were determined by calculating the proportion of each class comprised by individual small molecule metabolites.

Statistical analyses were conducted using World Programming System software (World Programming Ltd., Hampshire, United Kingdom). Significance was defined as a P value less than or equal to 0.05. Red blood cell index values (hematocrit, packed cell volume, hemoglobin, and red blood cell counts) and serum small molecule metabolite concentrations (total and individual classes) from Month 1, 3, and 6 were compared with Month 0 for both modified diet and baseline diet dolphins using ANOVA contrasts.

### Red Blood Cell Index Values

Comparisons of red blood cell index values in bottlenose dolphins (Tursiops truncatus) fed a Modified Diet versus baseline diet over 6 months. The Modified Diet is described above. Table 7 (depicted graphically in FIG. 1) provides data on improving hematocrit among individual dolphins while on the Modified Diet.

TABLE 7

| | Month 0 | | Month 1 | | Month 3 | | Month 6 | |
|---|---|---|---|---|---|---|---|---|
| | Modified | Baseline | Modified | Baseline | Modified | Baseline | Modified | Baseline |
| Variable | Diet | Diet | Diet | Diet | Diet | Diet | Diet | Diet |
| Hemoglobin | 13 ± 1 | 13 ± 1 | 14 ± 1 | 13 ± 1 | 15 ± 1* | 14 ± 1 | 15 ± 1* | 13 ± 1 |
| Hematocrit | 44 ± 5 | 44 ± 3 | 46 ± 3* | 44 ± 3 | 47 ± 2* | 45 ± 2 | 47 ± 2* | 44 ± 2 |
| RDW | 15 ± 2 | 15 ± 2 | 14 ± 1 | 15 ± 2 | 13 ± 1* | 15 ± 2 | 13 ± 1* | 16 ± 3 |
| RBC | 3.0 ± 0.3 | 3.0 ± 0.2 | 3.2 ± 0.2 | 3.0 ± 0.2 | 3.4 ± 0.2* | 3.2 ± 0.2 | 3.3 ± 0.3* | 3.1 ± 0.2 |
| MCV | 145 ± 5 | 145 ± 5 | 144 ± 5 | 143 ± 5 | 142 ± 5 | 141 ± 5 | 141 ± 5 | 142 ± 6 |
| MCH | 44 ± 1 | 44 ± 2 | 44 ± 2 | 44 ± 2 | 44 ± 2* | 43 ± 2 | 44 ± 2 | 43 ± 2 |
| MCHC | 31 ± 1 | 30 ± 1 | 31 ± 0.8 | 30 ± 1 | 31 ± 1* | 30 ± 1 | 31 ± 1* | 30 ± 1 |
| NRBC | 1.8 ± 2.9 | 1.4 ± 2.5 | 0.5 ± 0.9* | 1.2 ± 1.5 | 0.2 ± 0.6* | 1.1 ± 1.0 | 0.2 ± 0.5 | 0.7 ± 1.3 |
| PCV | 38 ± 4 | 38 ± 3 | 41 ± 2* | 39 ± 2 | 42 ± 2* | 40 ± 3 | 42 ± 2* | 39 ± 2 |

### Primary Human Cell Phenotypic Profiling

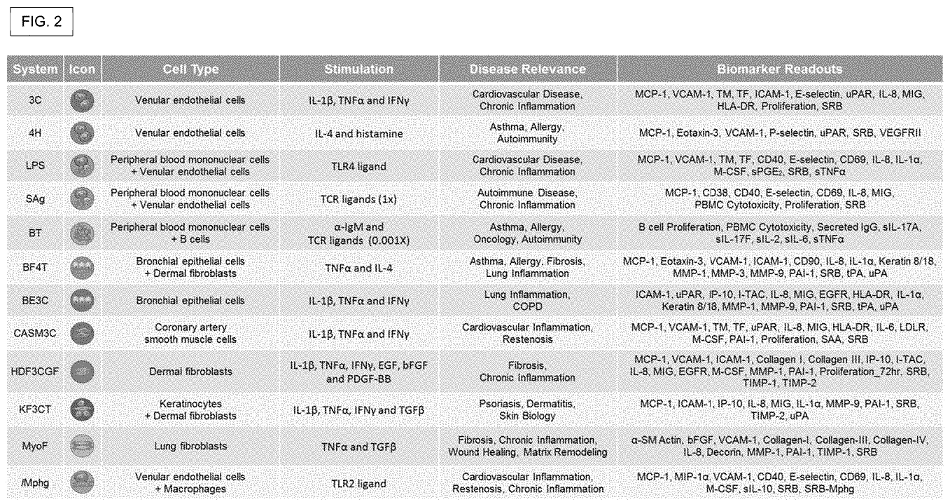

It was hypothesized that, given the compounds' associations with lower risks of comorbidities of aging in long-lived dolphins, the compounds of the embodiments would have relevant activities in human cell systems mimicking conditions related to the quality of aging and longevity. To test this hypothesis, 12 primary human cell systems from the BioMAP Diversity PLUS® panel (Eurofins/DiscoverX, Fremont, Calif.) mimicking inflammation and fibrosis relevant to chronic inflammation, liver disease, respiratory disease, skin disease, wound healing, scarring, allergies, asthma, autoimmune disease, cancer, and cardiovascular disease, were exposed to pure, synthetic forms of the selected compounds at one or two concentrations (6.7 and/or 20 μM). Quantitative measurements of 148 biomarker activities across this broad panel, along with comparative analysis of biological activities from known bioactive agents, were used to predict and compare the efficacy and function of the targeted compounds across these systems compared to non-treated control systems. Activated BioMAP systems were incubated with the targeted compounds for 24 to 72 hours. Biomarker activities were considered significant when biomarker values were outside of the 95^{th} percentile significance envelope and had at least one concentration with an effect size >20% (log 10 ratio) >0.1. FIG. 2 describes the different cell systems.

This study demonstrated that the targeted compounds at 6.7 to 20 μM significantly reduced numerous biomarkers of both Th1 and Th2 type inflammation and fibrosis across multiple human cell systems mimicking chronic inflammation, liver disease, respiratory disease, skin disease, wound healing, scarring, allergies, asthma, autoimmune disease, cancer, and cardiovascular disease. Table 8 provides a summary of disease biomarkers significantly changed across 12 primary human cell systems mimicking various disease states and treated with selected compounds compared to non-treated control systems (significant=biomarker values of treated systems outside of the 95^{th} percentile significance envelope and had at least one concentration with an effect size >20% (log 10 ratio) >0.1 compared to non-treated controls). A summary of compounds by disease and tissue relevance is provided in Table 9. The summary is of relevant diseases and tissues treated with selected compounds at 6.7 to 20 μM, based on primary human cell phenotypic profiling.

Key conclusions from the experiment included the following. While initial dolphin studies demonstrated associations between higher blood levels of selected compounds and lower risks of conditions related to the quality of aging and longevity, this study demonstrated direct disease-attenuating activities by our selected compounds in primary human cell systems mimicking chronic inflammation, liver disease, respiratory disease, skin disease, wound healing, scarring, allergies, asthma, autoimmune disease, cancer, and cardiovascular disease. These disease-attenuating activities were present at compound concentrations ranging from 6.7 to 20 μM.

TABLE 8

| | | | | Log₁₀ ratio | |
|---|---|---|---|---|---|
| | | | | of treated vs. | |
| | Human Cell | Disease/tissue | Affected | non-treated | |
| Compound | System | relevance | biomarker | systems | Relevance of biomarker |
| 4-guanidino- | 4H | Autoimmunity, | MCP-1 | −0.12 | Inflammation activity modeling Th2 vascular |
| butanoate | | Allergy, Asthma | | | inflammation |
| | SAg | Chronic | CD38 | −0.10 | Inflammation activity modeling T cell-driven Th1 |
| | | inflammation, | | | vascular inflammation |
| | | cardiovascular | Proliferation | −0.13 | T cell proliferation |
| | | disease | | | |
| | BT | Asthma, oncology, | sIgG | −0.49 | Inflammation-related activity modeling T cell |
| | | autoimmunity, | IL-17A | −0.17 | dependent B cell activation |
| | | allergy | TNF-α | −0.10 | |
| | HDF3CGF | Chronic | VCAM1 | −0.19 | Inflammation-related activity modeling Th1 |
| | | inflammation, | | | inflammation involved in wound healing and matrix |
| | | fibrosis | | | remodeling of skin |
| | | | Collagen III | −0.23 | Pro-fibrotic activity |
| | | | CXCL10 | −0.11 | Inflammation-related activity modeling Th1 |
| | | | | | inflammation involved in wound healing and matrix |
| | | | CXCL11 | −0.19 | remodeling of skin |
| | | | Proliferation | −1.09 | Pro-fibrotic activity |
| | | | TIMP-1 | −0.14 | Inflammation-related activity modeling Th1 |
| | | | | | inflammation involved in wound healing and matrix |
| | | | | | remodeling of skin |
| | MyoF | Wound healing, | Collagen IV | 0.13 | Major structural component of the basal lamina to |
| | | fibrosis, chronic | | | repair/remodel tissue |
| | | inflammation | | | |
| Chenodoxy- | SAg | Chronic | CXCL8 | −0.10 | Inflammation-related activity modeling T cell-driven |
| cholate | | inflammation, | | | Th1 vascular inflammation |
| | | cardiovascular | | | |
| | | disease | | | |
| Cholate | BE3C | COPD, lung | MMP-1 | −0.10 | Tissue remodeling activity modeling Th1 lung |
| | | inflammation | | | inflammation |
| Gamma- | 3C | Cardiovascular | Tissue factor | −0.12 | Promotes the formation of thrombin during process of |
| glutamyl- | | disease, chronic | | | vascular thrombosis modeling Th1 vascular |
| histidine | | inflammation | | | inflammation |
| | LPS | Chronic | CD40 | −0.11 | Inflammatory activity modeling monocyte-driven Th1 |
| | | inflammation, | E-selectin | −0.11 | vascular inflammation |
| | | cardiovascular | | | |
| | | disease | | | |
| | BT | Asthma, oncology, | TNF-α | −0.18 | Inflammation-related activity modeling T cell |
| | | autoimmunity, | | | dependent B cell activation |
| | | allergy | | | |
| | BE3C | COPD, lung | MMP-1 | −0.10 | Tissue remodeling activity modeling Th1 lung |
| | | inflammation | | | inflammation |
| | CASM3C | Cardiovascular | Proliferation | 0.11 | Measure of coronary artery smooth muscle cell |
| | | inflammation, | | | proliferation |
| | | restenosis | | | |
| | /Mphg | Chronic | CXCL8 | −0.15 | Inflammation-related activity modeling macrophage- |
| | | inflammation, | | | driven Th1 vascular inflammation |
| | | restenosis, | | | |
| | | cardiovascular | | | |
| | | disease | | | |
| Hippurate | 4H | Autoimmunity, | P-selectin | −0.10 | Inflammation-related activity modeling Th2 vascular |
| | | Allergy, Asthma | | | inflammation |
| | LPS | Chronic | CD40 | −0.11 | Inflammatory activity modeling monocyte-driven Th1 |
| | | inflammation, | | | vascular inflammation |
| | | cardiovascular | | | |
| | | disease | | | |
| | BE3C | COPD, lung | MMP-1 | −0.11 | Tissue remodeling activity modeling Th1 lung |
| | | inflammation | | | inflammation |
| | CASM3C | Cardiovascular | Proliferation | −0.10 | Measure of coronary artery smooth muscle cell |
| | | inflammation, | | | proliferation |
| | | restenosis | | | |
| | IMphg | Chronic | CXCL8 | −0.12 | Inflammation-related activity modeling macrophage- |
| | | inflammation, | | | driven Th1 vascular inflammation |
| | | restenosis, | | | |
| | | cardiovascular | | | |
| | | disease | | | |
| Myo-inositol | 4H | Autoimmunity, Allergy, | MCP-1 | −0.13 | Inflammation activity modeling Th2 vascular |
| | | Asthma | | | inflammation |
| | SAg | Chronic inflammation, | Proliferation | −0.20 | T cell proliferation |
| | | cardiovascular disease | | | |
| | BT | Asthma, oncology, | sIgG | −0.62 | Inflammation-related activity modeling T cell |
| | | autoimmunity, allergy | sIL-17A | −0.12 | dependent B cell activation |
| | | | sIL-17F | −0.15 | |
| | | | sIL-2 | −0.16 | |
| | | | sTNF-α | −0.21 | |
| | BF4T | Fibrosis, lung inflammation, | MCP-1 | −0.21 | Inflammation-related activity modeling Th2 airway |
| | | asthma, allergy | Eotaxin-3 | −0.19 | inflammation |
| | BE3C | COPD, lung inflammation | ICAM-1 | −0.11 | Inflammation-related activity modeling Th1 lung |
| | | | CXCL10 | −0.58 | inflammation |
| | | | CXCL11 | −0.28 | |
| | | | CXCL8 | −0.26 | |
| | | | EGFR | −0.11 | Tissue remodeling activity modeling Th1 lung |
| | | | HLA-DR | −0.10 | inflammation |
| | | | MMP-9 | −0.25 | |
| | | | PAI-1 | −0.18 | Pro-fibrotic activity |
| | KF3CT | Dermatitis, psoriasis | CXCL10 | −0.26 | Inflammation-related activity modeling Th1 cutaneous |
| | | | IL-1α | −0.11 | inflammation |
| | | | MMP-9 | −0.18 | Tissue remodeling activity modeling Th1 cutaneous |
| | | | PAI-1 | −0.27 | inflammation |
| | MyoF | Wound healing, fibrosis, | VCAM-1 | −0.11 | Inflammation-related activity modeling pulmonary |
| | | chronic inflammation | Collagen III | −0.11 | myofibroblast development |
| | | | | | Fibrotic activity |
| | | | Collagen IV | 0.24 | Major structural component of the basal lamina to |
| | | | | | repair/remodel tissue |
| | /Mphg | Chronic inflammation, | IL-1α | −0.10 | Inflammation-related activity modeling macrophage- |
| | | restenosis, cardiovascular | | | driven Th1 vascular inflammation |
| | | disease | | | |
| N-acetyl- | 4H | Autoimmunity, Allergy, | VEGFR2 | −0.16 | Receptor involved in endothelial cell proliferation and |
| threonine | | Asthma | | | vascular permeability modeling Th2 vascular |
| | | | | | inflammation |
| | BT | Asthma, oncology, | sIgG | −0.41 | Inflammation-related activity modeling T cell |
| | | autoimmunity, allergy | | | dependent B cell activation |
| Vanillyl- | /Mphg | Chronic inflammation, | CXCL8 | −0.18 | Inflammation-related activity modeling macrophage- |
| mandelate | | restenosis, | | | driven Th1 vascular inflammation |
| (VMA) | | cardiovascular disease | | | |
| Xanthosine | LPS | Chronic inflammation, | Tissue factor | −0.13 | Promotes formation of thrombin during process of |
| | | cardiovascular disease | | | thrombosis in system modeling monocyte-driven Th1 |
| | | | | | vascular inflammation |
| | | | CD40 | −0.12 | Inflammatory activity modeling monocyte-driven Th1 |
| | | | | | vascular inflammation |
| | /Mphg | Chronic inflammation, | CXCL8 | −0.13 | Inflammation-related activity modeling macrophage- |
| | | restenosis, | | | driven Th1 vascular inflammation |
| | | cardiovascular disease | | | |
| S-adenosyl- | 3C | Cardiovascular disease, | Proliferation | −0.27 | Measure of endothelial cell proliferation |
| homocysteine | | chronic inflammation | | | |
| (SAH) | SAg | Chronic inflammation, | Proliferation | −0.21 | T cell proliferation |
| | | cardiovascular disease | | | |
| | BT | Asthma, oncology, | sIgG | −0.29 | Inflammation-related activity modeling T cell dependent B cell |
| | | autoimmunity, allergy | | | activation |
| | BE3C | COPD, lung | CXCL11 | −0.13 | Inflammation-related activity modeling Th1 lung inflammation |
| | | inflammation | CXCL8 | −0.13 | |
| | | | HLA-DR | −0.13 | |
| | | | MMP-9 | −0.24 | Tissue remodeling activity modeling Th1 lung inflammation |
| | HDF3CGF | Chronic inflammation, | MCP-1 | −0.14 | Inflammation-related activity modeling Th1 inflammation |
| | | fibrosis | VCAM-1 | −0.17 | involved in wound healing and matrix remodeling of skin |
| | | | Collagen III | −0.21 | Fibrotic activity |
| | | | CXCL10 | −0.19 | Inflammation-related activity modeling Th1 inflammation |
| | | | CXCL11 | −0.25 | involved in wound healing and matrix remodeling of skin |
| | | | M-CSF | −0.19 | |
| | | | PAI-1 | −0.23 | Fibrotic activity |
| | | | Proliferation | −1.10 | |
| | | | TIMP-1 | −0.14 | Inflammation-related activity modeling Th1 inflammation |
| | | | | | involved inwound healing and matrix remodeling of skin |
| | KF3CT | Dermatitis, psoriasis | MMP-9 | −0.10 | Tissue remodeling activity modeling Th1 cutaneous |
| | | | PAI-1 | −0.28 | inflammation |
| | MyoF | Wound healing, fibrosis, | Alpha-SM | −0.18 | Activated myofibroblast marker in system modeling pulmonary |
| | | chronic inflammation | actin | | myofibroblast development |
| | | | VCAM-1 | −0.12 | Inflammation-related activity in system modeling pulmonary |
| | | | | | myofibroblast development |
| | | | Collagen I | −0.15 | Pro-fibrotic activity |
| | | | Collagen III | −0.27 | |
| | | | Collagen IV | 0.31 | Major structural component of the basal lamina to repair/ |
| | | | | | remodel tissue |

TABLE 9

| | Human Cell Phenotypic Profiling-Evidence of Relevant Diseases/Tissues Treated by Selected Compounds | | | | | | | | | | |
|---|---|---|---|---|---|---|---|---|---|---|---|
| | | | | | | | Skin | | | | |
| | Chronic | | | Liver | Respira- | Cardio- | Disease/ | | | Auto- | |
| Selected | inflammation | | Fibro- | disease | tory | vascular | Wound | Scarring | Asthma/ | immune | Can- |
| Compound | Th1 | Th2 | sis | Fibrosis | disease | disease | Healing | (fibrosis) | allergies | disease | cer |
| 4-guanidino- | x | x | x | x | x | x | x | x | x | x | x |
| butanoate | | | | | | | | | | | |
| Chenodoxy- | x | | | | | x | | | | | |
| cholate | | | | | | | | | | | |
| Cholate | x | | | | X | | | | | | |
| Gamma- | x | | | | x | x | | | x | x | X |
| glutamyl- | | | | | | | | | | | |
| histidine | | | | | | | | | | | |
| Hippurate | x | x | | | x | x | | | x | x | |
| Myo-inositol | x | x | x | x | x | x | x | x | x | x | X |
| N-acetyl- | | x | | | | | | | x | x | X |
| threonine | | | | | | | | | | | |
| S-adenosyl- | x | | x | x | x | x | x | x | x | x | X |
| homocysteine | | | | | | | | | | | |
| Vanillyl- | x | | | | | x | | | | | |
| mandelate | | | | | | | | | | | |
| Xanthosine | x | | | | | x | | | | | |

Small molecule (defined as less than 900 daltons in molecular weight) metabolites described herein can be present in serum due to either ingestion of food products or endogenous production. Thousands of small molecule metabolites can be detected and measured in serum of animals, including dolphins and humans. Metabolomics, the study of metabolites and their biologically relevant roles, is a relatively novel field of study. Due to the large number of metabolites present and high potential variation of metabolites driven by complex diets, environments, genetics, and long-term medications in human populations, identifying metabolites with the greatest biomarker and therapeutic potential has been complicated. Based upon the results using the methods of the embodiments, it can be proposed that small molecule metabolites described herein may be key players in detecting, preventing, and treating aging-associated conditions that impact quality of life and longevity.

To take advantage of these benefits, small molecule metabolites described herein can be used in a supplement, medical food, food additive, food fortifier, beverage additive, beverage fortifier, or pharmaceutical in any form, including as a tablet, encapsulated pill, gelcap pill, liquid suspension, spray, and powder. Additionally, diagnostic tests and assays for small molecule metabolites described herein in human and animal samples (including blood (serum, plasma, and erythrocyte membranes), urine, and feces) can be used to detect low small molecule metabolites and to continually monitor small molecule metabolite levels in patients. The use of small molecule metabolites can prevent, stem, and treat: aging and aging-associated conditions that impact quality of life and/or longevity, including inflammation, anemia, hyperglycemia, dyslipidemia, hyperinsulinemia, liver disease, iron overload, impaired skin integrity, wound healing, scarring, pain, allergies, sleep disorders and problems, and gastrointestinal disorders and problems, and other related conditions.

Methods and compositions related to or applicable to aging or related conditions are discussed in the following references, which are incorporated by reference herein in their entirety: Venn-Watson S, Parry C, Baird M, Stevenson S, Carlin K, Daniels R, Smith C R, Jones R, Wells R S, Ridgway S, Jensen E D (2015) Increased dietary intake of saturated fatty acid heptadecanoic acid (C17:0) associated with decreasing ferritin and alleviated metabolic syndrome in dolphins. PLOS ONE 10(7):e0132117, Collino S (2013) Metabolic signatures of extreme longevity in Northern Italian centenarians reveal a complex remodeling of lipids, amino acids, and gut microbiota metabolism. PLOS ONE 8:e56564, Siming M (2015) Organization of the mammalian metabolome according to organ function, lineage specialization, and longevity. Cell Metab 22:332-343, Cheng (2015) Distinct metabolomic signatures are associated with longevity in humans. Nat Comm doi:10.1039/ncomms7791, Gonzalez-Covarrubias V (2013) Lipidomics of familial longevity. Aging Cell 12:426-434, Montoliu I (2014) Serum profiling of healthy aging identifies phosphor- and sphingolipid species as markers of human longevity. Aging 6:9-25, Evans C (2010) NAD+ metabolite levels as a function of vitamins and calorie restriction: evidence for different mechanisms of longevity. BMC Chem Biol 10:2, Gonzalez-Covarrubias V (2013) Lipidomics in longevity and healthy aging. Biogerontol 14:663-672, Kristal B S (2005) Metabolomics: opening another window into aging. Sci Aging Know Environ 26:pe19.

In summary, a number of molecules discovered to have protective profiles against inflammation and other diseases of aging have demonstrated the ability to be increased in serum and to have these increases associated with attenuated diseases of aging. Different dolphin-based compounds may be selected as therapeutics based on specific diseases being targeted.

### Exemplary Pharmaceutical Compositions, Uses, and Methods

Pharmaceutical Composition 1: A pharmaceutical composition for treatment or prophylaxis of aging or an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, the pharmaceutical composition comprising: one or more small molecule biochemicals, or pharmaceutically acceptable salts, solvates, stereoisomers, or esters thereof; and a pharmaceutically acceptable carrier.

Pharmaceutical Composition 2: The pharmaceutical composition of Pharmaceutical Composition 1, wherein the one or more small molecule biochemicals is selected from the group consisting of amino acids, carbohydrates, lipids, nucleotides, peptides or xenobiotics, and combinations thereof.

Pharmaceutical Composition 3: The pharmaceutical composition of any of Pharmaceutical Composition 1 or 2, wherein the one or more small molecule biochemicals is capable of detection in serum at concentrations of from 1-10 nanomolar to 1-30 micromolar levels, has a low molecular weight of <900 daltons, and meets Lipinski's rule of five.

Pharmaceutical Composition 4: The pharmaceutical composition of any of Pharmaceutical Compositions 1 through 3, wherein the one or more small molecule biochemicals is an amino acid.

Pharmaceutical Composition 5: The pharmaceutical composition of Pharmaceutical Composition 4, wherein the amino acid is selected from the group consisting of 1-carboxyethylleucine, 1-carboxyethylvaline, 2-methylbutyrylcarnitine (C5), 3-hydroxyisobutyrate, 3-methyl-2-oxobutyrate, 3-methyl-2-oxovalerate, 4-guanidinobutanoate, 4-hydroxyglutamate, 4-methyl-2-oxopentanoate, 5-(galactosylhydroxy)-L-lysine, 5-oxoproline, betaine, creatine, indole-3-carboxylate, kynurenate, N6-acetyllysine, N-acetylalanine, N-acetylglycine, N-acetylisoleucine, N-acetylputrescine, N-acetyltaurine, N-acetylthreonine, pyroglutamine, vanillylmandelate (VMA), xanthurenate and combinations thereof.

Pharmaceutical Composition 6: The pharmaceutical composition of any of Pharmaceutical Compositions 1 through 3, wherein the one or more small molecule biochemicals is a peptide.

Pharmaceutical Composition 7: The pharmaceutical composition of Pharmaceutical Composition 6, wherein the peptide is selected from the group consisting of gamma-glutamylcitrulline, gamma-glutamylhistidine, and combinations thereof.

Pharmaceutical Composition 8: The pharmaceutical composition of any of Pharmaceutical Compositions 1 through 3, wherein the one or more small molecule biochemicals is a carbohydrate.

Pharmaceutical Composition 9: The pharmaceutical composition of Pharmaceutical Composition 8, wherein the carbohydrate is selected from the group consisting of arabitol/xylitol, lactate, pyruvate, ribitol, S-adenosylhomocysteine (SAH) and combinations thereof.

Pharmaceutical Composition 10: The pharmaceutical composition of any of Pharmaceutical Compositions 1 through 3, wherein the one or more small molecule biochemicals is a regulator of energy or a nucleotide.

Pharmaceutical Composition 11: The pharmaceutical composition of Pharmaceutical Composition 10, wherein the regulator of energy or the nucleotide is selected from the group consisting of aconitate [cis or trans], 2′-deoxycytidine, 2′-deoxyuridine, adenine, thymidine, xanthosine, and combinations thereof.

Pharmaceutical Composition 12: The pharmaceutical composition of any of Pharmaceutical Compositions 1 through 3, wherein the one or more small molecule biochemicals is a xenobiotic.

Pharmaceutical Composition 13: The pharmaceutical composition of Pharmaceutical Composition 12, wherein the xenobiotic is selected from the group consisting of benzoylcarnitine, erythritol, hippurate, methylnaphthyl sulfate, O-sulfo-L-tyrosine, and combinations thereof.

Pharmaceutical Composition 14: The pharmaceutical composition of any of Pharmaceutical Compositions 1 through 3, wherein the one or more small molecule biochemicals is a diacyglycerol.

Pharmaceutical Composition 15: The pharmaceutical composition of Pharmaceutical Composition 14, wherein the diacyglycerol is DAG (18:1/20:2).

Pharmaceutical Composition 16: The pharmaceutical composition of any of Pharmaceutical Compositions 1 through 3, wherein the one or more small molecule biochemicals is a sphingolipid.

Pharmaceutical Composition 17: The pharmaceutical composition of Pharmaceutical Composition 16, wherein the sphingolipid is lactosylceramide (LCER) 18:1

Pharmaceutical Composition 18: The pharmaceutical composition of any of Pharmaceutical Compositions 1 through 3, wherein the one or more small molecule biochemicals is a lipid.

Pharmaceutical Composition 19: The pharmaceutical composition of Pharmaceutical Composition 18, wherein the lipid is selected from the group consisting of chenodeoxycholate, cholate, maleate, myo-inositol, and combinations thereof.

Pharmaceutical Composition 20: The pharmaceutical composition of any of Pharmaceutical Compositions 1 through 3, wherein the one or more small molecule biochemicals is a phosphatidylethanolamine ester.

Pharmaceutical Composition 21: The pharmaceutical composition of Pharmaceutical Composition 20, wherein the phosphatidylethanolamine ester is selected from the group consisting of, PE (O-16:0/16:1), PE (O-18:0/20:1), and combinations thereof.

Pharmaceutical Composition 22: The pharmaceutical composition of any of Pharmaceutical Compositions 1 through 3, wherein the one or more small molecule biochemicals is a triacylglycerol.

Pharmaceutical Composition 23: The pharmaceutical composition of Pharmaceutical Composition 22, wherein the triacylglycerol is selected from the group consisting of TAG52:1-FA20:0, TAG52:2-FA20:0, TAG54:0-FA16:0, TAG54:1-FA18:1, TAG54:1-FA20:0, TAG54:2-FA20:0, TAG54:4-FA20:4, TAG55:6-FA20:3, TAG56:1-FA18:1, TAG56:2-FA20:0, TAG56:4-FA22:4, TAG58:5-FA18:1, TAG58:6-FA22:4, or TAG58:7-FA22:4, and combinations thereof.

Pharmaceutical Composition 24: The pharmaceutical composition of any of Pharmaceutical Compositions 1 through 3, wherein the one or more small molecule biochemicals is a free fatty acid.

Pharmaceutical Composition 25: The pharmaceutical composition of Pharmaceutical Composition 24, wherein the free fatty acid is selected from the group consisting of FFA 18:1, caprate (C10:0) and combinations thereof.

Pharmaceutical Composition 26: The pharmaceutical composition of any of Pharmaceutical Compositions 1 through 3, wherein the one or more small molecule biochemicals is a product of fatty acid metabolism.

Pharmaceutical Composition 27: The pharmaceutical composition of Pharmaceutical Composition 26, wherein the product of fatty acid metabolism is selected from the group consisting of propionylglycine, lignoceroylcarnitine (C24), cerotoylcarnitine (C26), N-palmitoylglycine, cis-4-decenoylcarnitine (C10:1), behenoylcarnitine (C22), pentadecanoylcarnitine (C15), arachidonoylcholine, and combinations thereof.

Pharmaceutical Composition 28: The pharmaceutical composition of any of Pharmaceutical Compositions 1 through 3, wherein the one or more small molecule biochemicals is a phosphatidylcholine.

Pharmaceutical Composition 29: The pharmaceutical composition of Pharmaceutical Composition 28, wherein the phosphatidylcholine is selected from the group consisting of PC (12:0/20:1), PC (20:0/16:1), PC (20:0/18:1), PC (20:0/18:2), PC (20:0/20:3), PC (20:0/20:4), PC (20:0/20:5) and combinations thereof.

Pharmaceutical Composition 30: The pharmaceutical composition of any of Pharmaceutical Compositions 1 through 3, wherein the one or more small molecule biochemicals is a phosphatidylethanolamine.

Pharmaceutical Composition 31: The pharmaceutical composition of Pharmaceutical Composition 30, wherein the phosphatidylethanolamine is selected from the group consisting of PE (14:0/20:1), PE (16:0/20:1), PE (16:0/22:4), PE (18:1/22:0), PE (18:1/22:4), PE (18:1/22:5) PE (18:1/22:6), and combinations thereof.

Pharmaceutical Composition 32: The pharmaceutical composition of any of Pharmaceutical Compositions 1 through 3, wherein the one or more small molecule biochemicals is a phosphatidylethanolamine plasmalogen.

Pharmaceutical Composition 33: The pharmaceutical composition of Pharmaceutical Composition 32, wherein the phosphatidylethanolamine plasmalogen is selected from the group consisting of PE (P-16:0/18:0), PE (P-18:0/18:1), PE (P-18:0/18:2), PE (P-18:0/22:2), PE (P-18:0/18:1), PE (P-18:2/22:6), and combinations thereof.

Pharmaceutical Composition 34: The pharmaceutical composition of any of Pharmaceutical Compositions 1 through 3, wherein the one or more small molecule biochemicals is a ceramide.

Pharmaceutical Composition 35: The pharmaceutical composition of Pharmaceutical Composition 34, wherein the ceramide is ceramide CER (14:0).

Pharmaceutical Composition 36: The pharmaceutical composition of any of Pharmaceutical Compositions 1 through 3, wherein the one or more small molecule biochemicals is a lipid metabolite.

Pharmaceutical Composition 37: The pharmaceutical composition of any of Pharmaceutical Compositions 1 through 3, wherein the small molecule biochemical is 4-guanidinobutanoate.

Pharmaceutical Composition 38: The pharmaceutical composition of any of Pharmaceutical Compositions 1 through 3, wherein the small molecule biochemical is gamma-glutamylhistidine.

Pharmaceutical Composition 39: The pharmaceutical composition of any of Pharmaceutical Compositions 1 through 3, wherein the small molecule biochemical is S-adenosylhomocysteine.

Pharmaceutical Composition 40: The pharmaceutical composition of any of Pharmaceutical Compositions 36 through 39, further comprising hippurate.

Pharmaceutical Composition 41: The pharmaceutical composition of any one of Pharmaceutical Compositions 1 through 40, wherein the composition is in a unit dosage form.

Pharmaceutical Composition 42: The pharmaceutical composition of any one of Pharmaceutical Compositions 1 through 41, configured for administration of from 2.5 mg to 50 mg, per 1 kg of body weight, of the one or more small molecule biochemicals or pharmaceutically acceptable salts thereof to a patient.

Pharmaceutical Composition 43: The pharmaceutical composition of any one of Pharmaceutical Compositions 1 through 42, configured for administration once per day.

Pharmaceutical Composition 44: The pharmaceutical composition of any one of Pharmaceutical Compositions 1 through 43, comprising from 0.01 mg to 10000 mg of the one or more small molecule biochemicals or pharmaceutically acceptable salt thereof.

Use 45: Use of a pharmaceutical composition of any one of Pharmaceutical Compositions 1 through 44, in the manufacture of a medicament for treatment or prophylaxis of aging or an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

Use 46: The use of Use 45, wherein the pharmaceutical composition is adapted to modulate a marker or a symptom of aging.

Use 47: The use of Use 45, wherein the pharmaceutical composition is adapted to modulate a marker or a symptom of an aging-related condition negatively impacting longevity or quality of life.

Use 48: The use of Use 45, wherein the pharmaceutical composition is adapted to modulate a marker or a symptom of an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

Use 49: The use of Use 45, wherein the pharmaceutical composition is adapted to modulate a factor that drives or exacerbates an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

Use 50: The use of Use 45, wherein the pharmaceutical composition is adapted to treat a condition selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems.

Use 52: The use of any one of Uses 45 through 50, wherein the condition is asthma.

Use 52: The use of any one of Uses 45 through 50, wherein the condition is an autoimmune disease.

Use 53: T The use of any one of Uses 45 through 50, wherein the condition is cardiovascular disease.

Use 54: The use of any one of Uses 45 through 53, wherein the marker is selected from the group consisting of serum concentration of the one or more small molecule biochemicals, plasma concentration of the one or more small molecule biochemicals, cell concentration of the one or more small molecule biochemicals, and tissue concentration of the one or more small molecule biochemicals.

Use 55: The use of any one of Uses 45 through 54, wherein the pharmaceutical composition is configured to increase a concentration of the one or more small molecule biochemicals in any one of serum, plasma, or a red blood cell membrane by from 1.1 to 6 times a patient's baseline concentration and/or to a concentration greater than 0.5 μM and less than 30 μM.

Method 56: A method treatment or prophylaxis of aging or an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, the method comprising: administering to a patient in need thereof, an effective amount of one or more small molecule biochemicals, or pharmaceutically acceptable salts, solvates, stereoisomers, or esters thereof.

Method 57: The method of Method 56, wherein the one or more small molecule biochemicals is selected from the group consisting of amino acids, carbohydrates, lipids, nucleotides, peptides, xenobiotics, and combinations thereof.

Method 58: The method of any one of Methods 56 through 57, wherein the one or more small molecule biochemicals is capable of detection in serum at concentrations of from 1-10 nanomolar to 1-10 micromolar levels, has a low molecular weight of <900 daltons, and meets Lipinski's rule of five.

Method 59: The method of any one of Methods 56 through 58, wherein the one or more small molecule biochemicals is an amino acid.

Method 60: The method of Method 59, wherein the amino acid is selected from the group consisting of 1-carboxyethylleucine, 1-carboxyethylvaline, 2-methylbutyrylcarnitine (C5), 3-hydroxyisobutyrate, 3-methyl-2-oxobutyrate, 3-methyl-2-oxovalerate, 4-guanidinobutanoate, 4-hydroxyglutamate, 4-methyl-2-oxopentanoate, 5-(galactosylhydroxy)-L-lysine, 5-oxoproline, betaine, creatine, indole-3-carboxylate, kynurenate, N6-acetyllysine, N-acetylalanine, N-acetylglycine, N-acetylisoleucine, N-acetylputrescine, N-acetyltaurine, N-acetylthreonine, pyroglutamine, vanillylmandelate (VMA), xanthurenate and combinations thereof.

Method 61: The method of any of Methods 56 through 58, wherein the one or more small molecule biochemicals is a peptide.

Method 62: The method of Method 61, wherein the peptide is selected from the group consisting of gamma-glutamylcitrulline, gamma-glutamylhistidine, and combinations thereof.

Method 63: The method of any of Methods 56 through 58, wherein the one or more small molecule biochemicals is a carbohydrate.

Method 64: The method of Method 63, wherein the carbohydrate is selected from the group consisting of arabitol/xylitol, lactate, pyruvate, ribitol, S-adenosylhomocysteine (SAH) and combinations thereof.

Method 65: The method of any of Methods 56 through 58, wherein the one or more small molecule biochemicals is a regulator of energy or a nucleotide.

Method 66: The method of Method 65, wherein the regulator of energy or the nucleotide is selected from the group consisting of aconitate [cis or trans], 2′-deoxycytidine, 2′-deoxyuridine, adenine, thymidine, xanthosine, and combinations thereof.

Method 67: The method of any of Methods 56 through 58, wherein the one or more small molecule biochemicals is a xenobiotic.

Method 68: The method of any of Method 67 wherein the xenobiotic is selected from the group consisting of benzoylcarnitine, erythritol, hippurate, methylnaphthyl sulfate, O-sulfo-L-tyrosine, and combinations thereof.

Method 69: The method of any of Methods 56 through 58, wherein the one or more small molecule biochemicals is a diacyglycerol.

Method 70: The method of Method 69, wherein the diacyglycerol is DAG (18:1/20:2).

Method 72: The method of any of Methods 56 through 58, wherein the one or more small molecule biochemicals is a sphingolipid.

Method 72: The method of Method 71, wherein the sphingolipid is lactosylceramide (LCER) 18:1

Method 73: The method of any of Methods 56 through 58, wherein the one or more small molecule biochemicals is a lipid.

Method 74: The method of Method 73, wherein the lipid is selected from the group consisting of chenodeoxycholate, cholate, maleate, myo-inositol, and combinations thereof.

Method 75: The method of any of Methods 56 through 58, wherein the one or more small molecule biochemicals is a phosphatidylethanolamine ester.

Method 76: The method of Method 75, wherein the phosphatidylethanolamine ester is selected from the group consisting of, PE (0-16:0/16:1), PE (0-18:0/20:1), and combinations thereof.

Method 77: The method of any of Methods 56 through 58, wherein the one or more small molecule biochemicals is a triacylglycerol.

Method 78: The method of Method 77, wherein the triacylglycerol is selected from the group consisting of TAG52:1-FA20:0, TAG52:2-FA20:0, TAG54:0-FA16:0, TAG54:1-FA18:1, TAG54:1-FA20:0, TAG54:2-FA20:0, TAG54:4-FA20:4, TAG55:6-FA20:3, TAG56:1-FA18:1, TAG56:2-FA20:0, TAG56:4-FA22:4, TAG58:5-FA18:1, TAG58:6-FA22:4, or TAG58:7-FA22:4, and combinations thereof.

Method 79: The method of any of Methods 56 through 58, wherein the one or more small molecule biochemicals is a free fatty acid.

Method 80: The method of Method 79, wherein the free fatty acid is selected from the group consisting of FFA 18:1, caprate (C10:0) and combinations thereof.

Method 82: The method of any of Methods 56 through 58, wherein the one or more small molecule biochemicals is a product of fatty acid metabolism.

Method 82: The method of Method 81, wherein the product of fatty acid metabolism is selected from the group consisting of propionylglycine, lignoceroylcarnitine (C24), cerotoylcarnitine (C26), N-palmitoylglycine, cis-4-decenoylcarnitine (C10:1), behenoylcarnitine (C22), pentadecanoylcarnitine (C15), arachidonoylcholine, and combinations thereof.

Method 83: The method of any of Methods 56 through 58, wherein the one or more small molecule biochemicals is a phosphatidylcholine.

Method 84: The method of Method 83, wherein the phosphatidylcholine is selected from the group consisting of PC (12:0/20:1), PC (20:0/16:1), PC (20:0/18:1), PC (20:0/18:2), PC (20:0/20:3), PC (20:0/20:4), PC (20:0/20:5) and combinations thereof.

Method 85: The method of any of Methods 56 through 58, wherein the one or more small molecule biochemicals is a phosphatidylethanolamine.

Method 86: The method of Method 85, wherein the phosphatidylethanolamine is selected from the group consisting of PE (14:0/20:1), PE (16:0/20:1), PE (16:0/22:4), PE (18:1/22:0), PE (18:1/22:4), PE (18:1/22:5) PE (18:1/22:6), and combinations thereof.

Method 87: The method of any of Methods 56 through 58, wherein the one or more small molecule biochemicals is a phosphatidylethanolamine plasmalogen.

Method 88: The method of Method 87, wherein the phosphatidylethanolamine plasmalogen is selected from the group consisting of PE (P-16:0/18:0), PE (P-18:0/18:1), PE (P-18:0/18:2), PE (P-18:0/22:2), PE (P-18:0/18:1), PE (P-18:2/22:6), and combinations thereof.

Method 89: The method of any one of Methods 56 through 58, wherein the one or more small molecule biochemicals is an amino acid or lipid.

Method 90: The method of Method 56, wherein the small molecule biochemical is 4-guanidinobutanoate.

Method 91: The method of Method 56, wherein the small molecule biochemical is gamma-glutamylhistidine.

Method 92: The method of Method 56, wherein the small molecule biochemical is S-adenosylhomocysteine.

Method 93: The method of Method 56, wherein the pharmaceutical composition further comprises hippurate.

Method 94: The method of any one of Methods 56 through 93, wherein the pharmaceutical composition comprises a plurality of different small molecule biochemicals.

Method 95: The method of any one of Methods 56 through 94, wherein the one or more small molecule biochemicals or pharmaceutically acceptable salts, solvates, stereoisomers, or esters thereof is provided as a pharmaceutical composition in a unit dosage form comprising the one or more small molecule biochemicals or pharmaceutically acceptable salts thereof and a pharmaceutically acceptable carrier.

Method 96: The method of any one of Methods 56 through 95, wherein the unit dosage form comprises from 0.01 mg to 10000 mg of the one or more small molecule biochemicals or pharmaceutically acceptable salts thereof.

Method 97: The method of any one of Methods 56 through 96, wherein from 2.5 mg to 50 mg of the one or more small molecule biochemicals or pharmaceutically acceptable salts, solvates, stereoisomers, or esters thereof is administered to the patient, per 1 kg of body weight, per day.

Method 98: The method of any one of Methods 56 through 97, wherein the one or more small molecule biochemicals or pharmaceutically acceptable salts, solvates, stereoisomers, or esters thereof is administered to the patient once per day.

Method 99: The method of any one of Methods 56 through 98, wherein a serum, plasma, red blood cell, or tissue concentration is increased between 1.1 and 6 times a patient's baseline concentration and/or to a concentration greater than 0.5 μM and less than 30 μM.

Method 100: The method of any one of Methods 56 through 99, wherein the condition is asthma.

Method 101: The method of any one of Methods 56 through 99, wherein the condition is an autoimmune disease.

Method 102: The method of any one of Method 56 through 99, wherein the condition is cardiovascular disease.

Composition 103: A composition substantially as described herein.

Use 104: A use substantially as described herein.

Method 105: A method substantially as described herein.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of treatment or prophylaxis of aging or an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, the method comprising:
administering to a patient in need thereof, an effective amount of one or more small molecule biochemicals, or pharmaceutically acceptable salts, solvates, stereoisomers, or esters thereof.

2. The method of claim 1, wherein the one or more small molecule biochemicals is selected from the group consisting of amino acids, carbohydrates, lipids, nucleotides, peptides, xenobiotics, and combinations thereof.

3. The method of claim 1, wherein the one or more small molecule biochemicals is capable of detection in serum at concentrations of from 1-10 nanomolar to 1-10 micromolar levels, has a low molecular weight of <900 daltons, and meets Lipinski's rule of five.

4. The method of claim 1, wherein the one or more small molecule biochemicals is an amino acid selected from the group consisting of the amino acid is selected from the group consisting of 4-guanidinobutanoate, 5-(galactosylhydroxyl)-L-lysine, N-acetylthreonine, S-adenosylhomocysteine (SAH), vanillylmandelate (VMA), xanthosine, and combinations thereof.

5. The method of claim 1, wherein the one or more small molecule biochemicals is 4-guanidinobutanoate.

6. The method of claim 1, wherein the one or more small molecule biochemicals is gamma-glutamylhistidine.

7. The method of claim 1, wherein the one or more small molecule biochemicals is S-adenosylhomocysteine.

8. The method of claim 1, further comprising administering hippurate, and wherein the one or more small molecule biochemicals is selected from the group consisting of 4-guanidinobutanoate, gamma-glutamylhistidine, and S-adenosylhomocysteine.

9. The method of claim 1, wherein the condition is asthma.

10. The method of claim 1, wherein the condition is an autoimmune disease.

11. The method of claim 1, wherein the condition is cardiovascular disease.

12. The method of claim 1, wherein a serum, plasma, red blood cell, or tissue concentration of the one or more small molecule biochemicals is increased by from 1.1 to 6 times of the patient's baseline concentration and/or to a concentration greater than 0.5 μM and less than 30 μM.

13. The method of claim 1, wherein the one or more small molecule biochemicals or pharmaceutically acceptable salts, solvates, stereoisomers, or esters thereof is administered in a form selected from the group consisting of a foodstuff, a nutritional supplement, and a pharmaceutical composition in a unit dosage form.

14. The method of claim 13, wherein the unit dosage form comprises from 0.01 mg to 10000 mg of the one or more small molecule biochemicals or pharmaceutically acceptable salts thereof.

15. The method of claim 1, wherein from 2.5 mg to 50 mg of the one or more small molecule biochemicals or pharmaceutically acceptable salts, solvates, stereoisomers, or esters thereof is administered to the patient, per 1 kg of body weight, per day.

16. A pharmaceutical composition for treatment or prophylaxis of aging or an aging-related condition negatively impacting longevity or quality of life, wherein the condition is selected from the group consisting of asthma, autoimmune disease, cancer, cardiovascular disease, inflammation, anemia, hyperglycemia, dyslipidemia, hyperinsulinemia, liver disease, iron overload, impaired skin integrity, wound healing, scarring, pain, allergies, respiratory diseases, sleep disorders, sleep problems, gastrointestinal disorders, and gastrointestinal problems, the pharmaceutical composition comprising:
one or more small molecule biochemicals, or pharmaceutically acceptable salts, solvates, stereoisomers, or esters thereof; and
a pharmaceutically acceptable carrier.

17. The pharmaceutical composition of claim 16, wherein the one or more small molecule biochemicals is selected from the group consisting of amino acids, carbohydrates, lipids, a regulator of energy, nucleotides, peptides, xenobiotics, diaglycerols, sphingolipids, phosphatidylethanolamine esters, triacylglycerols, and combinations thereof.

18. The pharmaceutical composition of claim 16, wherein the one or more small molecule biochemicals is capable of detection in serum at concentrations of from 1-10 nanomolar to 1-30 micromolar levels, has a low molecular weight of <900 daltons, and meets Lipinski's rule of five.

19. The pharmaceutical composition of claim 16, wherein the one or more small molecule biochemicals is selected from the group consisting of 4-guanidinobutanoate, 5-(galactosylhydroxyl)-L-lysine, N-acetylthreonine, S-adenosylhomocysteine (SAH), vanillylmandelate (VMA), xanthosine, and combinations thereof, and combinations thereof.

20. The pharmaceutical composition of claim 16, wherein the small molecule biochemical is selected from the group consisting of 4-guanidinobutanoate, gamma-glutamylhistidine, and S-adenosylhomocysteine, wherein the pharmaceutical composition further comprises hippurate.
